(19) United States
(12) Patent Application Publication
(10) Pub. No.: US 2026/0162766 A1
(43) Pub. Date: Jun. 11, 2026

(54) GENOMIC SCORE PREDICTIVE OF IMMUNOTHERAPY RESPONSE

(71) Applicant: Ohio State Innovation Foundation, Columbus, OH (US)

(72) Inventor: Vineeth Sukrithan, Columbus, OH (US)

(21) Appl. No.: 19/270,091

(22) Filed: Jul. 15, 2025

Related U.S. Application Data
(60) Provisional application No. 63/671,439, filed on Jul. 15, 2024.

(51) Int. Cl. G16B 20/20; G16B 20/10; G16B 40/20

(52) U.S. Cl. CPC G16B 20/20; G16B 20/10; G16B 40/20

(57) ABSTRACT

Disclosed herein is a novel genomic scoring system that can predict immune checkpoint inhibitors response based on the identification of gene alterations in a panel of genes using gene sequencing of compared to wildtype controls.

### CROSS-REFERENCE TO RELATED APPLICATIONS

This application claims benefit of U.S. Provisional Application No. 63/671,439, filed Jul. 15, 2024, which is hereby incorporated herein by reference in its entirety.

### BACKGROUND OF THE INVENTION

Immune checkpoint therapies can yield durable responses and long-lasting survival benefit across some cancer types. Indeed, checkpoint therapies have been approved for use in metastatic melanoma, non-small cell lung cancer, bladder cancer, and renal cell carcinoma, including as a first-line therapy for non-small cell lung cancer. However, many subjects among a population of subjects having the same cancer type do not exhibit a therapeutic benefit or relapse despite being treated with the same immune checkpoint therapy. It is presently unclear which factors associated with a cancer or type thereof, such as mutational load, neoantigen presentation, transcriptomic signatures, microbiome features, immune cell infiltration, or other indicators, are predictive of response to immune checkpoint therapies. Accordingly, there remains a great need in the art to identify biomarkers predictive of immune checkpoint therapy in order to better treat cancer of subjects in need thereof.

### SUMMARY OF THE INVENTION

Disclosed herein is a genomic scoring system based on a search of available literature and review of patients with neuroendocrine neoplasms (NENs) treated with immune checkpoint inhibitors (ICI) at a large tertiary academic medical center to study the predictive ability of the genomic score in NENs.

In particular, disclosed herein is a method of treating a solid tumor in a subject that involves first gene sequencing a tumor sample from the subject to identify gene alterations compared to wildtype controls in at least 70, 71, 72, 73, 74, 75, 76, 77. 78, 79, 80, 81, 82, 83, 84, 85, 86, 87, 88, 89, 90, 95, 100, 105, 110, 115, 120, 125, 130, 135, 140, 145, 150, 155, 160, 161, or 162 genes selected from the group consisting of AMER1, APC, ARID1A, ARID1B, ARID2, ASXL1, ATM, ATR, ATRX, AURKA, AXIN1, AXIN2, B2M, BABAM1, BAP1, BCL2, BCL6, BCOR, BLM, BMPR1A, BRCA2, BRD4, BRIP1, CARM1, CD273, CD274, CDK12, CDK6, CDK8, CHEK1, CHEK2, CSF1R, CTNNB1, CUL3, DDR2, DICER, DNMT1, DNMT3A, DNMT3B, DOT1L, DROSHA, EGFR, EIF1AX, EIF4A2, EP300, ERBB3, ERBB4, ERCC5, ERRF12, ESR1, ETV1, ETV6, FANCA, FANCB, FANCC, FANCD2, FANCL, FBXW7, FGFR3, FGFR4, FLT3, FLT4, HGF, HIST1H1C, HIST1H1E, HLA-A, HRAS, IL7R, INHBA, INPP4B, INSR, IRF4, IRS1, JAK1, JAK2, JUB, KDR, KEAP1, KMT2A, KMT2B, KMT2C, KMT2D, KRAS, LATS1, MAP3K1, MAX, MED12, MGA, MLH1, MRE11A, MSH2, MSH3, MSH6, MST1, MST1R, MUTYH, MYC, MYCL, MYCN, NBN, NF1, NFE2L2, NFKBIA, NOTCH, NOTCH1, NOTCH2, NOTCH3, NOTCH4, NTRK1, NTRK3, PAK7, PBRM1, PDCD1, PDGFRA, PDGFRB, PIK3C2G, PIK3C3, PIK3CG, PIK3R3, PMS1, PMS2, POLD1, POLE, PPM1D, PTCH1, PTEN, PTPRD, PTPRO, PTPRS, PTPRT, RAD50, RARA, RASA1, RBM10, RECQL, REL, RET, RICTOR, RNF43, RPS6KB2, SETD2, SF3B1, SH2D1A, SMAD4, SMARCA4, SMARCB1, SMARCD1, SMO, SOCS1, STK11, TBX3, TCF7L2, TET2, TGFBR1, TGFBR2, TNFRSF14, TRAF7, U2AF1, VHL, WT1, XPO1, and ZFHX3, obtaining a survival score of at least 2x from the scoring below (where x is any integer); and treating the subject with an immunotherapy.

A survival score can be calculated from the gene alterations based on the following scheme:
the presence of an amino acid substitution in a gene selected from the group consisting of ATR, ATM, POLE, and POLD1 increases the survival score by 2x (where x is any number, such as for each gene alteration;
the presence of an amino acid substitution in a gene selected from the group consisting of ATRX, AJUBA, AKT2, ARID1A, ARID2, ARIDIB, AURKA, BABAM1, BCL2, BCL6, BCOR, BLM, BMPR1A, BRCA2, BRD4, BRIP1, CARM1, CDK12, CDK6, CDK8, CHEK1, CHEK2, CSF1R, DICER, DOT1L, DROSHA, EGFR, EIF1AX, EIF4A2, EP300, ERBB3, ERBB4, ERCC5, ESR1, ETV1, ETV6, FANCA, FANCB, FANCC, FANCD2, FANCL, FBXW7, FGFR3, FGFR4, FLT3, FLT4, HGF, HIST1H1C, HIST1H1E, HRAS, INHBA, INPP4B, INSR, IRF4, IRS1, KDR, KMT2A, KMT2B, KMT2C, KMT2D, LATS1, MAP3K1, MAX, MED12, MGA, MLH1, MRE11A, MSH2, MSH3, MSH6, MST1, MST1R, MUTYH, NBN, NOTCH1, NOTCH2, NOTCH3, NOTCH4, NTRK1, NTRK3, MTRKR3, PAK7, PBRM1, PDCD1, PDGFRA, PDGFRB, PIK3C2G, PIK3C3, PIK3CG, PMS1, PMS2, PPM1D, PTCH1, PTPRD, PTPRO, PTPRS, PTPRT, RAD50, RARA, RASA1, RECQL, REL, RET, RPS6KB2, SETD2, SF3B1, SMARCA4, SMARCB1, SMARCD1, SMO, SOCS1, TBX3, TGFBR1, TGFBR2, U2AF1, VHL, XPO1, and ZFHX3, increases the survival score by 1x for each gene alteration;
amplification of PDL1 or PDL2 increases the survival score by 1x for each gene alteration;
the presence of an amino acid substitution in a gene selected from the group consisting of AKT1, AMER1, APC, ASXL1, AXIN1, AXIN2, BAP1, B2M, CD273, CD274, CTNNB1, CUL3, DNMT1, DNMT3A, DNMT3B, ERRFI2, HLA-A, IL7R, JAK1, JAK2, KEAP1, KRAS, MYC, MYCL, MYCN, NF1, NFE2L2, NFKBIA, PIK3R3, RNF43, SH2D1A, SMAD4, STK11, TCF7L2, TET2, TNFRSF14, and WT1 decreases the survival score by 1x for each gene alteration;
amplification of RICTOR decreases the survival score by 1x for each gene alteration;
deletion, loss, frameshift, or truncation of SH2D1A, increases the survival score by 2(x) for each gene alteration; and
deletion, loss, frameshift, or truncation of NOTCH, PTEN, and RBM10 decreases the survival score by one (1) for each gene alteration.

In some embodiments, the immunotherapy comprises a checkpoint inhibitor. For example, the checkpoint inhibitor can be an anti-PD-1 antibody, anti-PD-L1 antibody, anti-CTLA-4 antibody, or a combination thereof.

In some embodiments, the solid tumor is found in a subject with bladder cancer, breast cancer, colorectal cancer, esophagogastric cancer, glioma, head and neck cancer, melanoma, non-small cell lung cancer, renal cell carcinoma, or a combination thereof.

In some embodiments, the at least 70 genes sequenced in step (a) include at least 70 of the genes selected from the group consisting of AKT1, AKT2, AMER1, APC, ARID1A, ARID2, ATM, ATR, ATRX, AURKA, AXIN1, AXIN2, B2M, BABAM1, BAP1, BCL2, BLM, BMPR1A, BRCA2, BRD4, CARM1, CD274, CDK8, CDK12, CHEK1, CHEK2, CTNNB1, CUL3, DDR2, DNMT3A, DNMT3B, DOT1L, DROSHA, EGFR, EIF1AX, EIF4A2, EP300, ERBB3, ERBB4, ESR1, ETV1, ETV6, FANCA, FANCC, FBXW7, FGFR3, HIST1H1C, HLA-A, HRAS, INPP4B, INSR, IRF4, IRS1, JAK1, KMT2D, KRAS, MAP3K1, MLH1, MSH3, MSH6, MUTYH, MYC, MYCN, NF1, NFE2L2, NOTCH1, NOTCH4, NTRK1, PBRM1, PDCD1, PDGFRA, PIK3R3, PMS1, PMS2, POLD1, POLE, PTEN, PTPRS, RARA, RASA1, RBM10, RECQL, REL, SF3B1, SH2D1A, SMAD4, SMARCA4, SMARCB1, SOCS1, TBX3, TGFBR1, TGFBR2, TRAF7, U2AF1, WT1, and XPO1.

In some embodiments, the at least 70 genes sequenced in step (a) include at least AMER1, APC, ATR, ATRX, AXIN1, AXIN2, B2M, BAP1, BLM, BMPR1A, BRD4, CARM1, CD274, CDK8, CDK12, CHEK1, CHEK2, CTNNB1, DNMT3B, DOT1L, DROSHA, EGFR, EIF1AX, EIF4A2, EP300, ESR1, ETV1, ETV6, FBXW7, FGFR3, HIST1H1C, HLA-A, HRAS, INPP4B, IRS1, JAK1, KMT2D, KRAS, MLH1, MUTYH, MYC, MYCN, NFE2L2, NOTCH1, NTRK1, PDCD1, PIK3R3, PMS1, PMS2, POLD1, PTEN, RARA, RASA1, RBM10, RECQL, REL, SF3B1, SH2D1A, SMAD4, SMARCA4, SMARCB1, SOCS1, TBX3, TGFBR1, TGFBR2, TRAF7, WT1, and XPO1.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1 shows Kaplan-Meier estimate of Overall Survival in patients with neuroendocrine neoplasms treated with ICI.

FIG. 2 shows Kaplan-Meier estimate of Overall Survival in patients with neuroendocrine neoplasms treated with chemotherapy alone.

FIG. 3 shows Kaplan-Meier survival curve for all patients. The corresponding risk table is displayed below the plot.

FIG. 4 shows Kaplan-Meier survival curve for all patients stratified by gene score groups. The log-rank test yielded a p-value <0.001, indicating a statistically significant difference in survival distribution between gene score groups.

FIG. 5 shows Kaplan-Meier survival curve for patients with bladder cancer stratified by gene score groups. The p-value of the log-rank test was 0.057, indicating that there was no statistically significant difference in survival distribution between gene score groups for patients with bladder cancer.

FIG. 6 shows Kaplan-Meier survival curve for patients with breast cancer stratified by gene score groups. The log-rank test p-value was 0.344, indicating that there was no statistically significant difference in survival distribution between gene score groups for patients with breast cancer.

FIG. 7 shows Kaplan-Meier survival curve for patients with colorectal cancer stratified by gene score groups. The log-rank test yielded a p-value of 0.006, indicating a statistically significant difference in survival distribution between gene score groups for patients with colorectal cancer.

FIG. 8 shows Kaplan-Meier survival curve for patients with esophagogastric cancer stratified by gene score groups. The log-rank test p-value was 0.191, indicating that there was no statistically significant difference in survival distribution between gene score groups for patients with esophagogastric cancer.

FIG. 9 shows Kaplan-Meier survival curve for patients with glioma stratified by gene score groups. The p-value of the log-rank test was 0.947, indicating that there was no statistically significant difference in survival distribution between gene score groups for patients with glioma.

FIG. 10 shows Kaplan-Meier survival curve for patients with head and neck cancer stratified by gene score groups. The log-rank test yielded a p-value of 0.004, indicating a statistically significant difference in survival distribution between gene score groups for patients with head and neck cancer.

FIG. 11 shows Kaplan-Meier survival curve for patients with melanoma stratified by gene score groups. The log-rank test yielded a p-value of 0.001, indicating a statistically significant difference in survival distribution between gene score groups for patients with melanoma.

FIG. 12 shows Kaplan-Meier survival curve for patients with non-small cell lung cancer stratified by gene score groups. The log-rank test yielded a p-value of 0.004, indicating a statistically significant difference in survival distribution between gene score groups for patients with non-small cell lung cancer.

FIG. 13 shows Kaplan-Meier survival curve for patients with renal cell carcinoma stratified by gene score groups. The log-rank test yielded a p-value <0.001, indicating a statistically significant difference in survival distribution between gene score groups for patients with renal cell carcinoma.

FIG. 14 shows Kaplan-Meier survival curve for patients with cancer of unknown primary stratified by gene score groups. The log-rank test p-value is 0.404, indicating that there was no statistically significant difference in survival distribution between gene score groups for patients with cancer of unknown primary.

FIG. 15 shows Kaplan-Meier survival curve for patients with tumor mutation burden greater than 10 stratified by gene score groups. The log-rank test p-value was 0.008, indicating a statistically significant difference in survival distribution between gene score groups for patients with TMB>10.

FIG. 16 shows Kaplan-Meier survival curve for patients with tumor mutation burden smaller than 10 stratified by gene score groups. The log-rank test yielded a p-value <0.001, indicating a statistically significant difference in survival distribution between gene score groups for patients with TMB>10.

FIG. 17 shows a multivariate Cox model hazard ratios for overall survival using total gene score, tumor mutational burden (TMB), age group, sex, and cancer type as covariates. After fixing the TMB, age, sex and the cancer type, the gene score was significantly associated with the hazard of death. With one-unit increase in total gene score, the hazard of death was 7% lower. In contrast, TMB was not significantly associated with the hazard of death after adjusting for gene score, age, sex and the cancer type.

FIG. 18 shows the total gene score model for bladder cancer. Receiver operating characteristic (ROC) curves and the corresponding area under the curve (AUC) were generated at 24 months.

FIG. 19 shows the total gene score model for breast cancer. Receiver operating characteristic (ROC) curves and the corresponding area under the curve (AUC) were generated at 24 months.

FIG. 20 shows the total gene score model for colorectal cancer. Receiver operating characteristic (ROC) curves and the corresponding area under the curve (AUC) were generated at 24 months.

FIG. 21 shows the total gene score model for esophagogastric cancer. Receiver operating characteristic (ROC) curves and the corresponding area under the curve (AUC) were generated at 24 months.

FIG. 22 shows the total gene score model for glioma. Receiver operating characteristic (ROC) curves and the corresponding area under the curve (AUC) were generated at 24 months.

FIG. 23 shows the total gene score model for head and neck cancer. Receiver operating characteristic (ROC) curves and the corresponding area under the curve (AUC) were generated at 24 months.

FIG. 24 shows the total gene score model for melanoma. Receiver operating characteristic (ROC) curves and the corresponding area under the curve (AUC) were generated at 24 months.

FIG. 25 shows the total gene score model for non-small cell lung cancer. Receiver operating characteristic (ROC) curves and the corresponding area under the curve (AUC) were generated at 24 months.

FIG. 26 shows the total gene score model for renal cell carcinoma. Receiver operating characteristic (ROC) curves and the corresponding area under the curve (AUC) were generated at 24 months.

FIG. 27 shows the total gene score model for cancer of unknown primary. Receiver operating characteristic (ROC) curves and the corresponding area under the curve (AUC) were generated at 24 months.

FIG. 28 shows the TMB score model for bladder cancer. Receiver operating characteristic (ROC) curves and the corresponding area under the curve (AUC) were generated at 24 months.

FIG. 29 shows the TMB score model for breast cancer. Receiver operating characteristic (ROC) curves and the corresponding area under the curve (AUC) were generated at 24 months.

FIG. 30 shows the TMB score model for colorectal cancer. Receiver operating characteristic (ROC) curves and the corresponding area under the curve (AUC) were generated at 24 months.

FIG. 31 shows the TMB score model for esophagogastric cancer. Receiver operating characteristic (ROC) curves and the corresponding area under the curve (AUC) were generated at 24 months.

FIG. 32 shows the TMB score model for glioma. Receiver operating characteristic (ROC) curves and the corresponding area under the curve (AUC) were generated at 24 months.

FIG. 33 shows the TMB score model for head and neck cancer. Receiver operating characteristic (ROC) curves and the corresponding area under the curve (AUC) were generated at 24 months.

FIG. 34 shows the TMB score model for melanoma. Receiver operating characteristic (ROC) curves and the corresponding area under the curve (AUC) were generated at 24 months.

FIG. 35 shows the TMB score model for non-small cell lung cancer. Receiver operating characteristic (ROC) curves and the corresponding area under the curve (AUC) were generated at 24 months.

FIG. 36 shows the TMB score model for renal cell carcinoma. Receiver operating characteristic (ROC) curves and the corresponding area under the curve (AUC) were generated at 24 months.

FIG. 37 shows the TMB score model for cancer of unknown primary. Receiver operating characteristic (ROC) curves and the corresponding area under the curve (AUC) were generated at 24 months.

FIG. 38 shows the distribution of concordance indices from 1,000 Cox regression models after randomly dropping 40 genes from the gene list. The histogram illustrates the variability in model performance, with the mean and standard deviation of the concordance values indicated in the right lower corner of the plot. The lower bound of the 95% CI of the concordance indices was 0.572.

FIG. 39 shows the distribution of concordance indices from 1,000 Cox regression models after randomly dropping 50 genes from the gene list. The histogram illustrates the variability in model performance, with the mean and standard deviation of the concordance values indicated in the right lower corner of the plot. The lower bound of the 95% CI of the concordance indices was 0.566.

FIG. 40 shows the distribution of concordance indices from 1,000 Cox regression models after randomly dropping 60 genes from the gene list. The histogram illustrates the variability in model performance, with the mean and standard deviation of the concordance values indicated in the right lower corner of the plot. The lower bound of the 95% CI of the concordance indices was 0.562.

FIG. 41 shows the distribution of concordance indices from 1,000 Cox regression models after dropping 98 genes from the gene list based on ranking. The histogram illustrates the variability in model performance, with the mean and standard deviation of the concordance values indicated in the right lower corner of the plot. The lower bound of the 95% CI of the concordance indices was 0.568.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. In some embodiments, such cells exhibit such characteristics in part or in full due to the expression and activity of immune checkpoint proteins, such as PD-1, PD-L1, and/or CTLA-4. Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The “normal” copy number (e.g., germline and/or somatic) of a biomarker nucleic acid or “normal” level of expression of a biomarker nucleic acid or protein is the activity/level of expression or copy number in a biological sample, e.g., a sample containing tissue, whole blood, serum, plasma, buccal scrape, saliva, cerebrospinal fluid, urine, stool, and bone marrow, from a subject, e.g., a human, not afflicted with cancer, or from a corresponding non-cancerous tissue in the same subject who has cancer.

The term “determining a suitable treatment regimen for the subject” is taken to mean the determination of a treatment regimen (i.e., a single therapy or a combination of different therapies that are used for the prevention and/or treatment of the cancer in the subject) for a subject that is started, modified and/or ended based or essentially based or at least partially based on the results of the analysis according to the present invention. One example is determining whether to provide targeted therapy against a cancer to provide immunotherapy that generally increases immune responses against the cancer (e.g., immune checkpoint therapy). Another example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “immune checkpoint” refers to a group of molecules on the cell surface of CD4+ and/or CD8+ T cells that fine-tune immune responses by down-modulating or inhibiting an anti-tumor immune response. Immune checkpoint proteins are well known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein.

“Immune checkpoint therapy” refers to the use of agents that inhibit immune checkpoint nucleic acids and/or proteins. Inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g, the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and anti-CTLA-4 antibodies, either alone or in combination, are used to inhibit immune checkpoints.

The terms “gene alteration” and “genomic variant” are used interchangeably to refer to any variation in the DNA that results in an alteration in the protein sequence or expression. Gene alterations include genomic variations that results in amino acid substitutions, deletions, frameshift mutations, and truncations. Gene alterations also include gene deletions, i.e. loss of one or both alleles as well as gene copy number gains and amplification. Gene alterations also include mutations that are predicted to result in loss or gain of function of the protein or not yet known to have a functional significance, i.e. a variant of unknown significance (VUS).

As used herein, the term “area under the curve” or “AUC” refers to the area under the receiver operating characteristic curve (ROC) as is well known in the art. The area under the curve (AUC) measurements help compare the accuracy of the classifier via the overall data range. Classifiers with larger area under the curve (AUC) have greater ability to accurately classify an unknown between two groups of interest. In distinguishing between the two populations, the receiver operating characteristic curve (ROC) has the property of being used to represent a particular feature (e.g., any item of biomarker and/or additional biomedical information described in the present invention) in graphical form. Typically, the above feature data across the entire population (e.g., patient group and control group) is sorted in ascending order based on a single feature value. Then, for each value of the above-described features, a true positive rate and a false positive rate for the data are calculated. The true positive rate is determined by calculating the number of cases higher than or equal to a value for the characteristic thereof and dividing the number of cases by the total number of cases. The false positive rate is determined by counting the number of control groups above the value for the characteristic and dividing by the total number of control groups. Although the definition refers to the case where the characteristic of the patient group is high relative to the control group, the definition also applies to the case where the characteristic of the patient group is low relative to the control group (in this case, the number of samples whose values are lower than the above characteristic can be calculated). A receiver operating characteristic curve (ROC) may be generated for other single calculations, and also for a single characteristic, in order to provide a single sum value, e.g., two or more characteristics may be mathematically combined (e.g., added, subtracted, etc.), which may be represented by the receiver operating characteristic curve (ROC). Additionally, combinations of multiple characteristics that can derive a single calculated value can be plotted against a receiver operating characteristic curve (ROC). These combinations of characteristics may constitute tests. The receiver operating characteristic curve (ROC) is a graph showing the true positive rate (sensitivity) of the test relative to the false positive rate (1-specificity) of the test.

Each point on the ROC graph represents a sensitivity/1-specificity pair corresponding to a particular decision threshold. Tests with perfect discrimination (no overlap in the two result distributions) have ROC plots across the top left corner with a true positive score of 1.0 or 100% (full sensitivity) and a false positive score of 0 (full specificity). The theoretical plot for the test without discrimination (same distribution of results for both groups) is a 45° diagonal from the bottom left corner to the top right corner. Most of the figures are between these two extremes. If the ROC plot is well below the 45° diagonal, this is easily corrected by reversing the criteria for “positive” from “greater than” to “less than” or vice versa. Qualitatively, the closer the graph is to the upper left corner, the higher the overall accuracy of the test. Depending on the desired confidence interval, a threshold can be derived from the ROC curve, allowing a given event to be diagnosed with the appropriate balance of sensitivity and specificity, respectively.

### Genomic Scoring

### Subjects

In one embodiment, the subject for whom predicted likelihood of efficacy of an immune checkpoint therapy is determined, is a mammal (e.g., mouse, rat, primate, non-human mammal, domestic animal, such as a dog, cat, cow, horse, and the like), and is preferably a human.

In another embodiment of the methods of the present invention, the subject has not undergone treatment, such as chemotherapy, radiation therapy, targeted therapy, and/or immune checkpoint therapy. In still another embodiment, the subject has undergone treatment, such as chemotherapy, radiation therapy, targeted therapy, and/or immune checkpoint therapy.

In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

The methods of the present invention can be used to determine the responsiveness to anti-immune checkpoint therapies of a cancer. In one embodiment, the cancer is one for which an immune checkpoint therapy (e.g., anti-PD-1 blocking antibody, anti-PD-L1 blocking antibody, CTLA-4 blocking antibody, and the like) is FDA-approved for treatment, such as those described in the Examples. In one embodiment, the cancers are solid tumors, such as lung cancer such as non-small cell lung cancer, bladder cancer, melanoma such as metastatic melanoma, and/or renal cell carcinoma. In another embodiment, the cancer is an epithelial cancer such as, but not limited to, brain cancer (e.g., glioblastomas) bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In still other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, brenner, or undifferentiated. In yet other embodiments, the cancer is a mesenchymal cancer, such as sarcoma.

### Sample Collection, Preparation and Separation

The sample from the subject is typically from a diseased tissue, such as cancer cells or tissues. Biological samples can be collected from a variety of sources from a patient including a body fluid sample, cell sample, or a tissue sample comprising nucleic acids. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In a preferred embodiment, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In one embodiment, the sample is serum, plasma, or urine. In another embodiment, the sample is serum.

### Gene Sequencing

Sequencing can be accomplished through classic Sanger sequencing methods, which are known in the art. In a preferred embodiment sequencing can be performed using high-throughput sequencing methods some of which allow detection of a sequenced nucleotide immediately after or upon its incorporation into a growing strand, for example, detection of sequence in substantially real time or real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read (or 500-1,000 bases per read for 454).

High-throughput sequencing methods can include but are not limited to, Massively Parallel Signature Sequencing (MPSS, Lynx Therapeutics), Polony sequencing, 454 pyrosequencing, Illumina (Solexa) sequencing, SOLID sequencing, on semiconductor sequencing, DNA nanoball sequencing, Helioscope™ single molecule sequencing, Single Molecule SMRT™ sequencing, Single Molecule real time (RNAP) sequencing, Nanopore DNA sequencing, and/or sequencing by hybridization, for example, a non-enzymatic method that uses a DNA microarray, or microfluidic Sanger sequencing.

In some embodiments, high-throughput sequencing can involve the use of technology available by Helicos BioSciences Corporation (Cambridge, Mass.) such as the Single Molecule Sequencing by Synthesis (SMSS) method. SMSS is unique because it allows for sequencing the entire human genome in up to 24 hours. This fast sequencing method also allows for detection of a SNP/nucleotide in a sequence in substantially real time or real time. Finally, SMSS is powerful because, like the MIP technology, it does not use a pre-amplification step prior to hybridization. SMSS does not use any amplification. SMSS is described in US Publication Application Nos. 2006/0024711; 2006/0024678; 2006/0012793; 2006/0012784; and 2005/0100932, which are incorporated by reference for these methods. In some embodiments, high-throughput sequencing involves the use of technology available by 454 Life Sciences, Inc. (a Roche company, Branford, Conn.) such as the PicoTiterPlate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

Gene sequencing is used herein to identify genomic variants in the identified genes. Genomic sequences within populations exhibit variability between individuals at many locations in the genome. For example, the human genome exhibits sequence variations that occur on average every 500 base pairs. Such genetic variations in nucleic acid sequences are commonly referred to as polymorphisms or polymorphic sites. As used herein, a polymorphism, e.g. genetic variation, includes a variation in the sequence of a gene in the genome amongst a population, such as allelic variations and other variations that arise or are observed. Thus, a polymorphism refers to the occurrence of two or more genetically determined alternative sequences or alleles in a population. These differences can occur in coding and non-coding portions of the genome, and can be manifested or detected as differences in nucleic acid sequences, gene expression, including, for example transcription, processing, translation, transport, protein processing, trafficking, DNA synthesis; expressed proteins, other gene products or products of biochemical pathways or in post-translational modifications and any other differences manifested amongst members of a population. A single nucleotide polymorphism (SNP) includes to a polymorphism that arises as the result of a single base change, such as an insertion, deletion or change in a base. A polymorphic marker or site is the locus at which divergence occurs. Such site can be as small as one base pair (an SNP). Polymorphic markers include, but are not limited to, restriction fragment length polymorphisms, variable number of tandem repeats (VNTR's), hypervariable regions, minisatellites, dinucleotide repeats, trinucleotide repeats, tetranucleotide repeats and other repeating patterns, simple sequence repeats and insertional elements, such as Alu. Polymorphic forms also are manifested as different mendelian alleles for a gene. Polymorphisms can be observed by differences in proteins, protein modifications, RNA expression modification, DNA and RNA methylation, regulatory factors that alter gene expression and DNA replication, and any other manifestation of alterations in genomic nucleic acid or organelle nucleic acids.

In some embodiments, a genetic variation can be a functional aberration that can alter gene function, gene expression, polypeptide expression, polypeptide function, or any combination thereof. In some embodiments, a genetic variation can be a loss-of-function mutation, gain-of-function mutation, dominant negative mutation, or reversion. In some embodiments, a genetic variation can be part of a gene's coding region or regulatory region. Regulatory regions can control gene expression and thus polypeptide expression. In some embodiments, a regulatory region can be a segment of DNA wherein regulatory polypeptides, for example, transcription factors, can bind. In some embodiments a regulatory region can be positioned near the gene being regulated, for example, positions upstream of the gene being regulated. In some embodiments, a regulatory region (e.g., enhancer element) can be several thousands of base pairs upstream or downstream of a gene.

In some embodiments, variants can include changes that affect a polypeptide, such as a change in expression level, sequence, function, localization, binding partners, or any combination thereof. In some embodiments, a genetic variation can be a frameshift mutation, nonsense mutation, missense mutation, neutral mutation, or silent mutation. For example, sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Such sequence changes can alter the polypeptide encoded by the nucleic acid, for example, if the change in the nucleic acid sequence causes a frame shift, the frame shift can result in a change in the encoded amino acids, and/or can result in the generation of a premature stop codon, causing generation of a truncated polypeptide. In some embodiments, a genetic variation associated with a neurological disorder can be a synonymous change in one or more nucleotides, for example, a change that does not result in a change in the amino acid sequence. Such a polymorphism can, for example, alter splice sites, affect the stability or transport of mRNA, or otherwise affect the transcription or translation of an encoded polypeptide. In some embodiments, a synonymous mutation can result in the polypeptide product having an altered structure due to rare codon usage that impacts polypeptide folding during translation, which in some cases may alter its function and/or drug binding properties if it is a drug target. In some embodiments, the changes that can alter DNA increase the possibility that structural changes, such as amplifications or deletions, occur at the somatic level. A polypeptide encoded by the reference nucleotide sequence can be a reference polypeptide with a particular reference amino acid sequence, and polypeptides encoded by variant nucleotide sequences can be variant polypeptides with variant amino acid sequences.

The most common sequence variants comprise base variations at a single base position in the genome, and such sequence variants, or polymorphisms, are commonly called single nucleotide polymorphisms (SNPs) or single nucleotide variants (SNVs). In some embodiments, a SNP represents a genetic variant present at greater than or equal to 1% occurrence in a population and in some embodiments a SNP can represent a genetic variant present at any frequency level in a population. A SNP can be a nucleotide sequence variation occurring when a single nucleotide at a location in the genome differs between members of a species or between paired chromosomes in a subject. SNPs can include variants of a single nucleotide, for example, at a given nucleotide position, some subjects can have a ‘G’, while others can have a ‘C’. SNPs can occur in a single mutational event, and therefore there can be two possible alleles possible at each SNP site; the original allele and the mutated allele. SNPs that are found to have two different bases in a single nucleotide position are referred to as biallelic SNPs, those with three are referred to as triallelic, and those with all four bases represented in the population are quadallelic. In some embodiments, SNPs can be considered neutral. In some embodiments SNPs can affect susceptibility to neurological disorders. SNP polymorphisms can have two alleles, for example, a subject can be homozygous for one allele of the polymorphism wherein both chromosomal copies of the individual have the same nucleotide at the SNP location, or a subject can be heterozygous wherein the two sister chromosomes of the subject contain different nucleotides. The SNP nomenclature as reported herein is the official Reference SNP (rs) ID identification tag as assigned to each unique SNP by the National Center for Biotechnological Information (NCBI).

Another genetic variation of the disclosure can be copy number variations (CNVs). As used herein, “CNVs” include alterations of the DNA of a genome that results an abnormal number of copies of one or more sections of DNA. In some embodiments, a CNV comprises a CNV-subregion. As used herein, a “CNV-subregion includes a continuous nucleotide sequence within a CNV. In some embodiments, the nucleotide sequence of a CNV-subregion can be shorter than the nucleotide sequence of the CNV. CNVs can be inherited or caused by de novo mutation and can be responsible for a substantial amount of human phenotypic variability, behavioral traits, and disease susceptibility. In a preferred embodiment, CNVs of the current disclosure can be associated with susceptibility to one or more neurological disorders, for example, Parkinson's Disease. In some embodiments, CNVs can include a single gene or include a contiguous set of genes. In some embodiments, CNVs can be caused by structural rearrangements of the genome, for example, translocations, insertions, deletions, amplifications, inversions, and interstitial deletions. In some embodiments, these structural rearrangements occur on one or more chromosomes. Low copy repeats (LCRs), which are region-specific repeat sequences, can be susceptible to these structural rearrangements, resulting in CNVs. Factors such as size, orientation, percentage similarity and the distance between the copies can influence the susceptibility of LCRs to genomic rearrangement. In some embodiments, CNVs are referred to as structural variants. In some embodiments, structural variants can be a broader class of variant that can also include copy number neutral alterations such as inversions and balanced translocations.

CNVs can account for genetic variation affecting a substantial proportion of the human genome, for example, known CNVs can cover over 15% of the human genome sequence (Estivill, X Armengol; L., PLOS Genetics 3:1787-99 (2007)). CNVs can affect gene expression, phenotypic variation and adaptation by disrupting gene dosage, and can cause disease, for example, microdeletion and microduplication disorders, and can confer susceptibility to diseases and disorders. Updated information about the location, type, and size of known CNVs can be found in one or more databases, for example, the Database of Genomic Variants (projects.tcag.ca/variation/), which currently contains data for over 66,000 CNVs (as of Nov. 2, 2010).

Other types of sequence variants can be found in the human genome and can be associated with a disease or disorder, including but not limited to, microsatellites. Microsatellite markers are stable, polymorphic, easily analyzed, and can occur regularly throughout the genome, making them especially suitable for genetic analysis. A polymorphic microsatellite can comprise multiple small repeats of bases, for example, CA repeats, at a particular site wherein the number of repeat lengths varies in a population. In some embodiments, microsatellites, for example, variable number of tandem repeats (VNTRs), can be short segments of DNA that have one or more repeated sequences, for example, about 2 to 5 nucleotides long, that can occur in non-coding DNA. In some embodiments, changes in microsatellites can occur during genetic recombination of sexual reproduction, increasing or decreasing the number of repeats found at an allele, or changing allele length.

### Immunotherapy

The efficacy of immunotherapy is predicted herein according to genomic variants in the disclosed genes. In some embodiments, immunotherapies, such as immune checkpoint inhibitors, can be administered once a subject is indicated as being a likely responder to immunotherapy based on the disclosed methods. In other embodiments, such immunotherapy can be avoided once a subject is indicated as not being a likely responder to immunotherapy and an alternative treatment regimen, such as untargeted anti-cancer therapies, can be administered. Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with immune checkpoint therapy.

In some embodiments, cancer immunotherapy comprises administration of an immune checkpoint inhibitor. In particular, the biomarkers of the present invention can accurately predict a response to such cancer immunotherapy of a subject.

In some embodiments, an immune checkpoint inhibitor comprises a PD-1 inhibitor or a PD-L1 inhibitor. Examples of PD-1 inhibitors include, but are not limited to, anti-PD-1 antibodies that inhibit an interaction between PD-1 and PD-L1 (e.g., binding) such as anti-PD-1 antibodies nivolumab and pembrolizumab. Examples of PD-L1 inhibitors include, but are not limited to, anti-PD-L1 antibodies that inhibit an interaction between PD-1 and PD-L1 (e.g., binding) such as anti-PD-L1 antibodies durvalumab, atezolizumab, and avelumab.

Immunotherapy can in some embodiments involve the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6 (5H)-phenanthridinone (Trevigen); benzamide (U.S. Pat. No. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454 (9); Herceg Z.; Wang Z.-Q. Mutation Research/Fundamental and Molecular Mechanisms of Mutagenesis, Volume 477, Number 1, 2 Jun. 2001, pp. 97-110 (14)). Poly (ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Genomic Score Predicts Survival with Immune Check-Point Blockade in Neuroendocrine Neoplasms

### Introduction

Immune checkpoint inhibitors (ICI) have been transformative for patients with multiple cancer types, including neuroendocrine neoplasms (NENs). The efficacy of the combination of nivolumab (anti-PD-1) and ipilimumab (anti-CTLA-4) was reported in a cohort of non-pancreatic NENs from the SWOG S1609 DART study. The ORR in patients with grade 3 NENs was 44% (Patel S P, et al. Clin Cancer Res. 2020 26 (10): 2290-6).

Subsequently, a prospectively defined cohort of 19 patients with high-grade NENs reported a 26% ORR, with an additional 5% of patients having stable disease for ≥6 months (Patel S P, et al. Cancer. 2021 127 (17): 3194-201). Another trial of nivolumab and ipilimumab among 29 patients had an ORR of 23% in Grade 2 and 31% in Grade 3 NENs (Klein O, et al. Clinical Cancer Research. 2020 26 (17): 4454-9). Beyond grade and differentiation, there is also evidence of increased activity in certain NEN primary sites such as lung (Yao J C, et al. Endocr Relat Cancer. 2021; Owen D H, et al. Clin Cancer Res. 2023 29 (4): 731-41). Toripalimab, an anti-PD-1 antibody, was tested in a phase Ib study of 40 patients with NENs who had Ki-67 expression in ≥10%. The majority had Neuroendocrine Carcinomas (NEC) ( 32/40; 80%). The objective response rate (ORR) was 20.0% ( 8/40). Patients with PD-L1 expression (≥10%) or high tumor mutational burden (TMB) had better ORR than PD-L1<10% (50.0% vs. 10.7%, p=0.019) and TMB-low patients (75.0% vs. 16.1%, P=0.03) (Lu M, et al. Clin Cancer Res. 2020 26 (10): 2337-45).

Overall, the National Comprehensive Cancer Network recommends Nivolumab and Ipilimumab in patients with Grade 3 NENs based on the results of the DART trial. However, predictors of response to ICI are lacking in NENs. There have been individual efforts to identify genomic correlates of response or lack-thereof to ICI in a pan-cancer fashion there are no validated genomic scores that can be used to predict survival (Keenan T E, et al. Nature Medicine. 2019 25 (3): 389-402). It's applicability in NENs is also poorly understood.

### Methods

This was a single-center, retrospective cohort study of patients with histologically confirmed neuroendocrine neoplasms at The Ohio State University Comprehensive Cancer Center (OSUCCC).

Included patients were required to be 18 years of age or older and have received at least one dose of ipilimumab and/or nivolumab. Protected patient populations such as pregnant women or prisoners were excluded.

Retrospective data were reviewed using the electronic medical record system. Baseline demographic information was obtained and included age, sex, ethnicity, baseline performance status, location of tumor, presence of metastases and treatment with prior radiation, chemoradiation and/or systemic chemotherapy. Where available, information was collected regarding PD-L1 status, tumor proportion score (TPS) and combined positive score (CPS). Tumor mutation burden (TMB) was also recorded along with all gene mutations found on commercially performed next generation sequencing (Foundation One or Tempus), including variants of unknown significance. A genomic score that encompasses both positive and negative predictors of survival was developed from a literature search (Table 1). The genomic score encompasses around 70 genes in 24 functional domains (Table 1). Eleven of these domains were negative in their impact on survival and a score of −1 was assigned per alteration. Certain gene alterations noted to have a particularly strong positive association with survival (DNA polymerase and DNA damage transduction) were assigned a score of +2 while all other genes predictive of a benefit were assigned a score of +1 per alteration.

TABLE 1

| Functional category | Genes Altered | Score (per alt.) |
|---|---|---|
| DNA Polymerases | POLE, POLD1 | +2 |
| DNA Damage transducers | ATR, ATM | +2 |
| Chromatin re-modelling/ | ATRX, BRD4, CARM1, DOT1L, EP300, | +1 |
| epigenetic functions | KMT2A, KMT2B, KMT2C, KMT2D, | |
| | SETD2 | |
| Microtubule Assembly | AURKA | +1 |
| RNA processing and | DICER, DROSHA, XPO1 | +1 |
| Export | | |
| Loss of Myeloid Cell | CSF1R | +1 |
| population | | |
| SWI/SNF sub-units | ARID1A, ARIDIB, ARID2, PBRM1, | +1 |
| | SMARCA4, SMARCB1, SMARCD1 | |
| Mis-match repair | MLH1, MSH2, MSH3, MSH6, PMS1, | +1 |
| | PMS2 | |
| DNA damage repair | BABAM1, BLM, BRCA2, BRIP1, CHEK1, | +1 |
| | CHEK2, ERCC5, FANCA, FANCB, | |
| | FANCC, FANCD2, FANCL, MRE11A, | |
| | MUTYH, NBN, RAD50, RECQL | |
| Sonic hedgehog signaling | PTCH1, SMO | +1 |
| Immune escape | CSF1R, PDCD1, ZFHX3 | +1 |
| Increased Lymphocyte | SH2D1A | +2 |
| Signaling through SLAM- | | |
| associated protein | | |
| Immune checkpoint | PDL1, PDL2 amplification | +1 |
| amplification | | |
| Loss of negative | PPM1D, PTPRD, PTPRO, PTPRS, | +1 |
| regulators of cytokine | PTPRT, SOCS1 | |
| signaling | | |
| Loss of degradation of | FBXW7 | +1 |
| NOTCH1/2 | | |
| Inhibition of tumor growth | AKT2, EGFR, ERBB3, ERBB4, ESR1, | +1 |
| signaling (truncating, non- | ETV1, FGFR3, FGFR4, FLT3, HGF, | |
| sense, frameshift, splice | HRAS, INPP4B, INSR, IRS1, IRF4, | |
| site and mis-sense that | MAP3K1, MST1, MST1R, NTRK1, | |
| are not predicted to be | NTRK3, NTRKR3, PDGFRA, PDGFRB, | |
| gain-of-function) | PIK3C2G, PIK3C3, PIK3CG, RASA1, | |
| | REL, RET, RPS6KB2 | |
| Inhibition of MYC | MGA, MAX | +1 |
| signaling | | |
| Inhibition of tumor | ETV6, FLT4, KDR, VHL | +1 |
| vascularization | | |
| Mutations in NOTCH | NOTCH1, NOTCH2, NOTCH3, NOTCH4 | +1 |
| pathway | | |
| Loss of TGFB signaling | BMPR1A, INHBA, PAK7, TGFBR1, | +1 |
| | TGFBR2 | |
| Spliceosome and reduced | U2AF1, SF3B1 | +1 |
| non-sense mediated | | |
| decay | | |
| Regulation of | BCOR, BCL2, BCL6, RARA | +1 |
| Transcription | | |
| RNA Polymerase II | CDK6, CDK8, CDK12, DICER, DROSHA, | +1 |
| transcription and | EIF1AX, EIF4A2, MED12, TBX3, XPO1 | |
| translation | | |
| Linker Histone | HIST1H1C, HIST1H1E | +1 |
| Increased YAP signaling | AJUBA, LATS1 | +1 |
| Histone post-translational | BAP1 | −1 |
| modification | | |
| Dysregulated RNA | RBM10 loss | −1 |
| splicing | | |
| Increased TGF-Beta | SMAD4 | −1 |
| signaling | | |
| Increased WNT signaling | CTNNB1, AMER1, APC, AXIN1, AXIN2, | −1 |
| | RNF43, TCF7L2 | |
| Reduced cytokine | JAK1, JAK2 alterations | −1 |
| signaling | | |
| Increase MYC signaling | MYC, MYCL, MYCN | −1 |
| Reduced checkpoint | CD274, CD273 | −1 |
| expression | | |
| Reduced NOTCH | NOTCH loss | −1 |
| pathway signaling | | |
| Increased mTOR | STK11, RICTOR amplification, PTEN | −1 |
| signaling and reduced | Damaging Mutation | |
| mitophagy | | |
| Increased anti-oxidant | KEAP1, CUL3, NFE2L2 | −1 |
| signaling | | |
| Increase myeloid cell | ASXL1, DNMT1, DNMT3A, DNMT3B, | −1 |
| population | TET2, WT1 | |
| Activators of Growth | AKT1, ERRFI2, KRAS, NF1, NFKBIA, | −1 |
| Signaling (Gain of | PIK3R3 | |
| Function or amplification | | |
| of growth protein or LOF | | |
| of regulatory/inhibitory | | |
| growth signaling) | | |
| Loss of MHC presentation | B2M, HLA-A, IL7R, SH2D1A | −1 |
| | (deletion/frameshift/loss/truncation), | |
| | TNFRSF14, TRAF7 | |

In addition, time points of treatment start, last treatment received, any radiographic progression and death were collected. Number of doses of nivolumab and ipilimumab, treatment interruptions, reason for discontinuation and treatment-related toxicities were also recorded. Patients received Nivolumab 240 mg IV every 2 weeks and Ipilimumab 1 mg/kg IV every 6 weeks. Patients had follow-up imaging assessments performed at intervals per the discretion of the treating clinician. Adverse effects will be graded using the National Cancer Institute's Common Terminology Criteria for Adverse Events version 5.0. Overall Survival (OS) is defined as the time from first dose of treatment to death.

### Results

A total of 54 patients met inclusion criteria. Median age was 60 (range 27-80). The primary site was colorectal (13%), pancreas (33%), lung (7%), small bowel/unknown (20%) and esophageal/gastric (9%). Most patients (87%) had grade 3. Morphology was well-differentiated in 35% and poorly differentiated in 63%.

The cohort was heavily pre-treated; 46% had progression on ≥3 prior lines of therapy. PD-L1 testing was positive in 9% ( 3/32) of patients with available samples. Median OS was 5.9 months (95% CI, 4.6-7.3] but 28% of patients survived for a year or more. ≥Grade 3 immune-related adverse events were seen in 14.8% and systemic steroids were administered in 26% of patients.

Complete and interpretable somatic NGS testing information was available in 85% ( 46/54) of patients and the association of GS-O with OS was studied in this population.

The median Tumor Mutational Burden (TMB) was not significantly different between GS-O<2 and ≥2 (3 vs. 3.5 muts/MB, p=0.87)

After adjusting for age, primary site, Ki-67%, differentiation, and TMB, GS-O was independently associated with overall survival (HR=0.19, p<0.001).

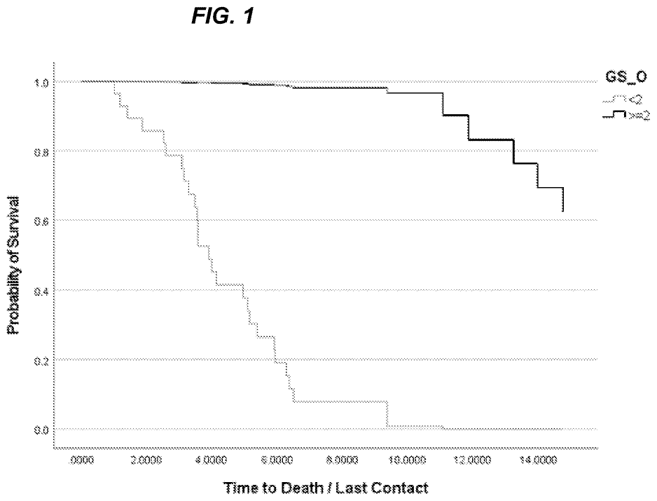

A GS-O≥2 was found in 37% ( 17/46). The median OS was not reached in patients with GS-O≥2 vs. 3.9 months in those with GS-O<2 (OR=0.007, univariate log-rank p<0.001) (FIG. 1).

Among patients with OS≥11 months, 100% (16/16) had a GS-O≥2, whereas among patients who died before 11 months, 0% (0/30) had a GS≥2 (Fisher's Exact test p<0.001).

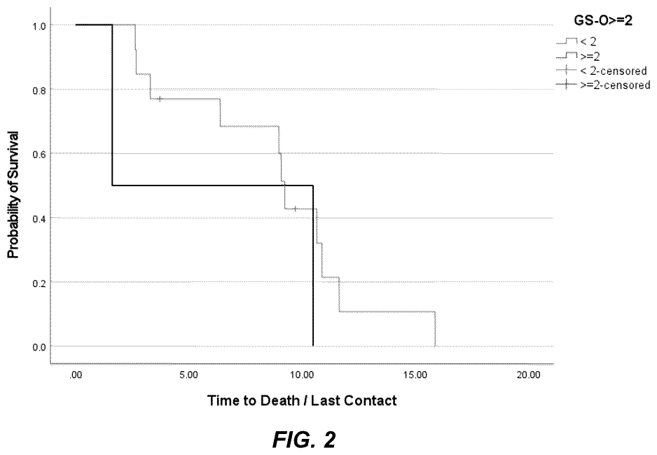

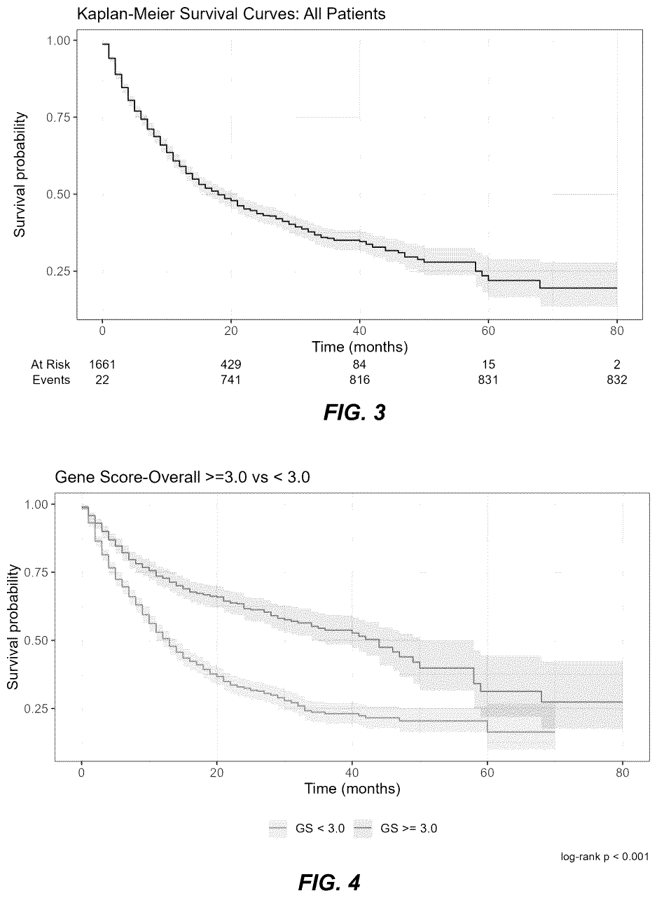

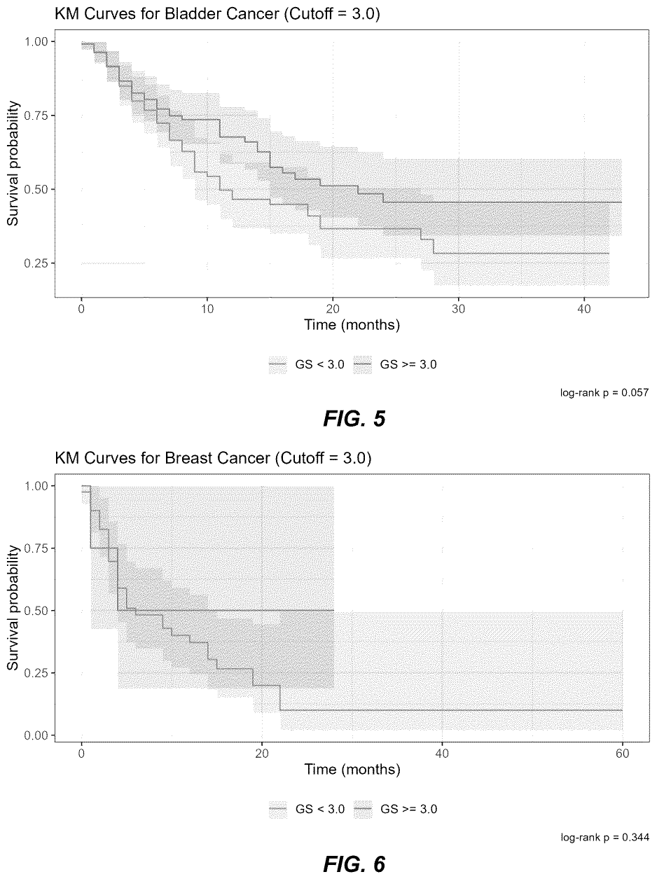

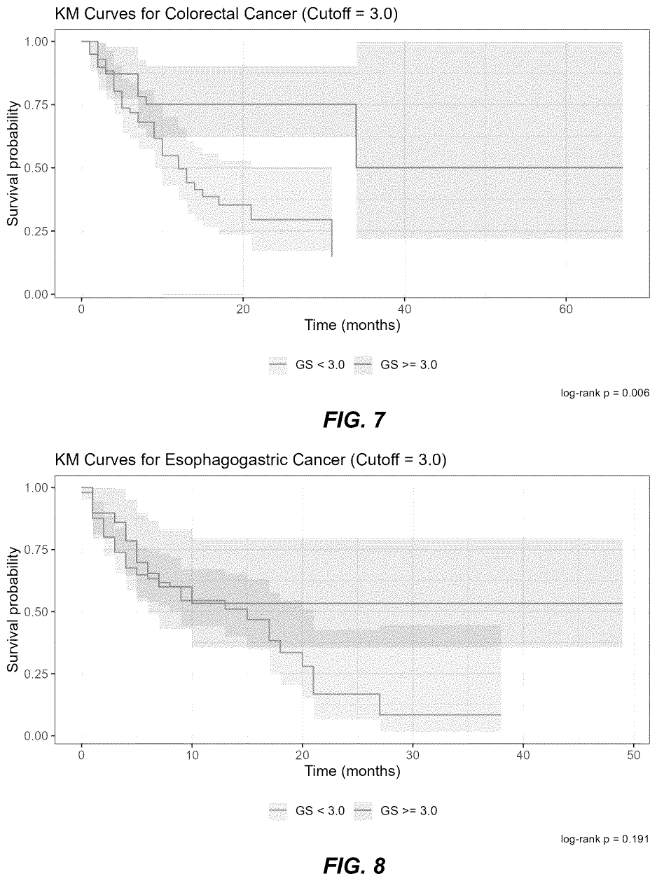

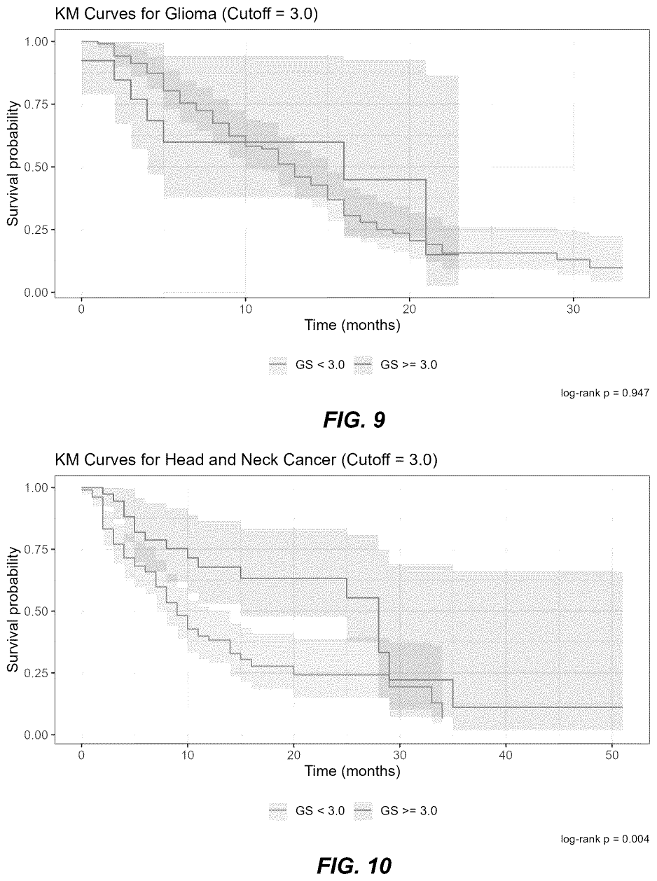

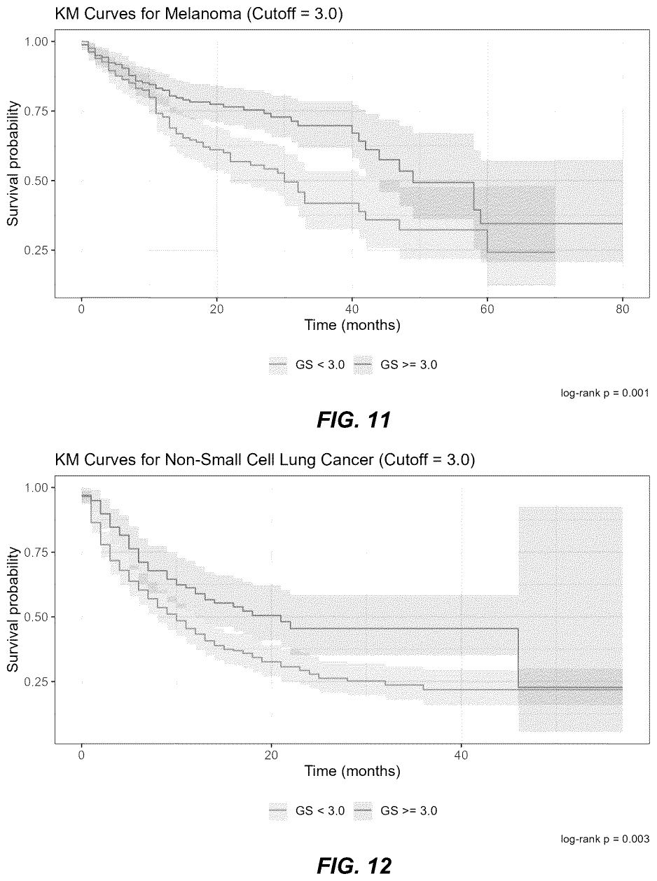

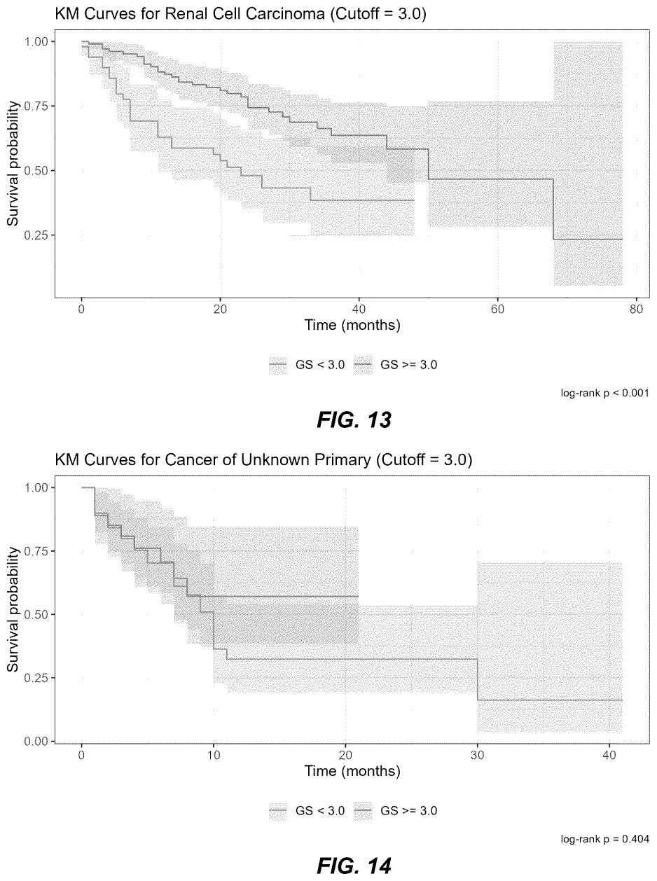

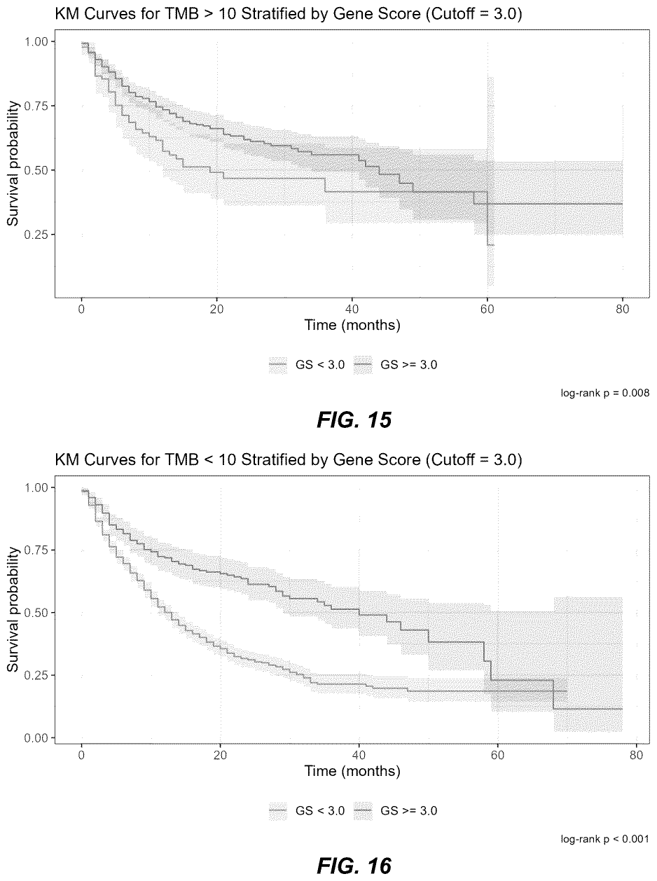

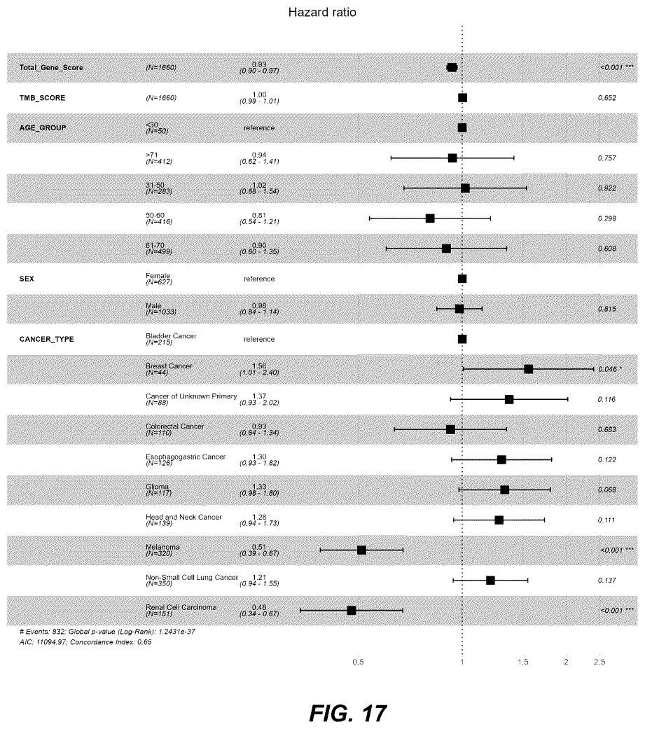

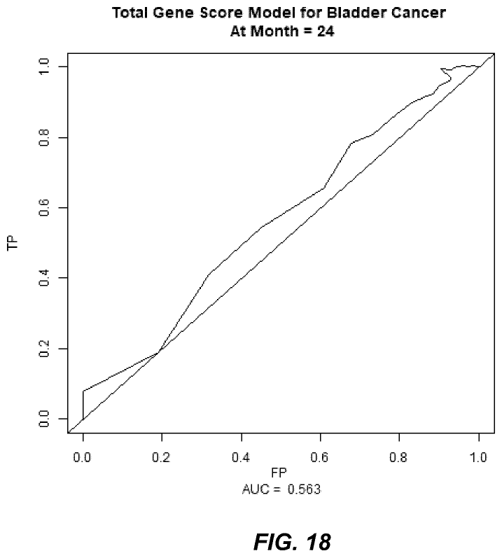

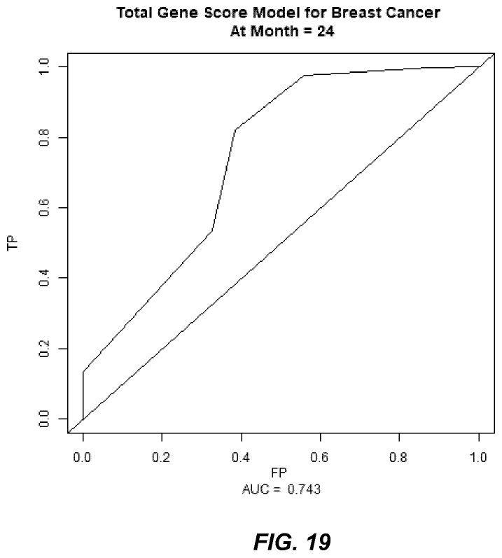

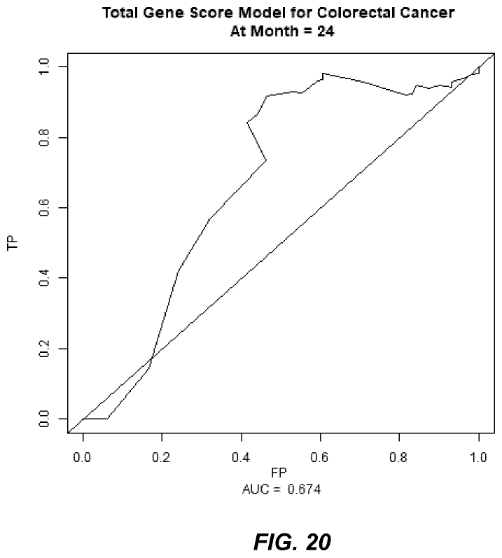

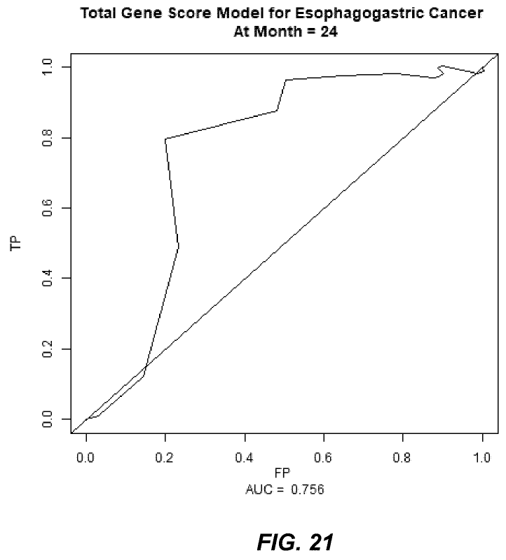

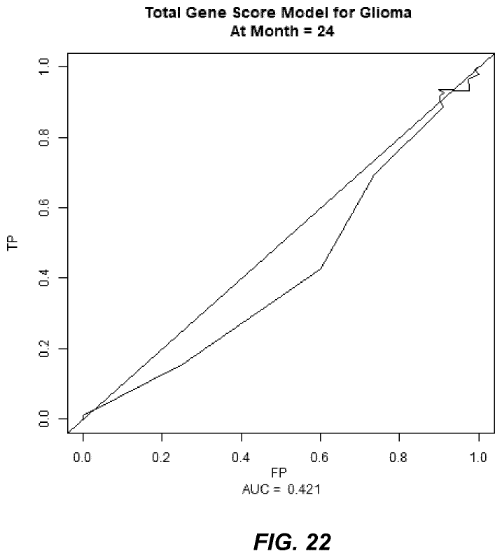

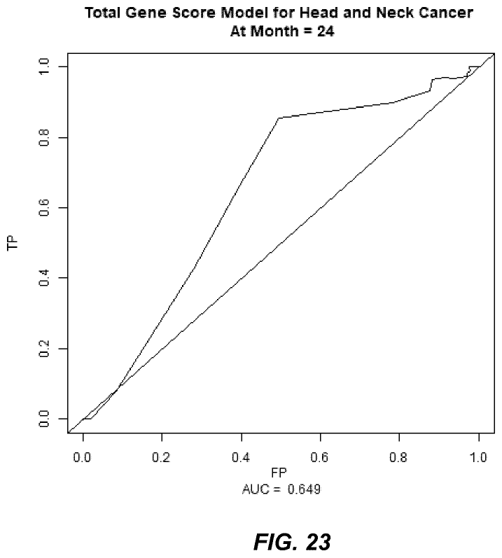

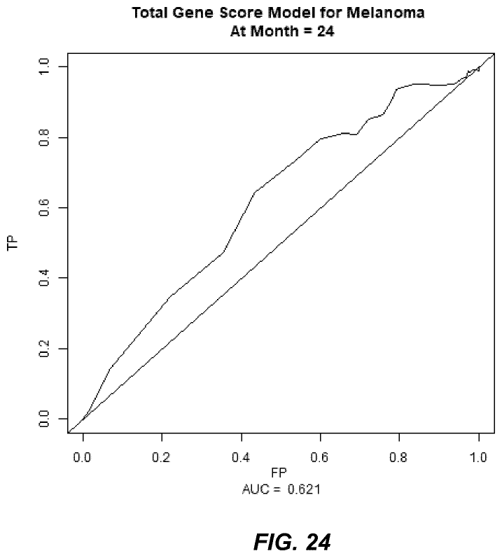

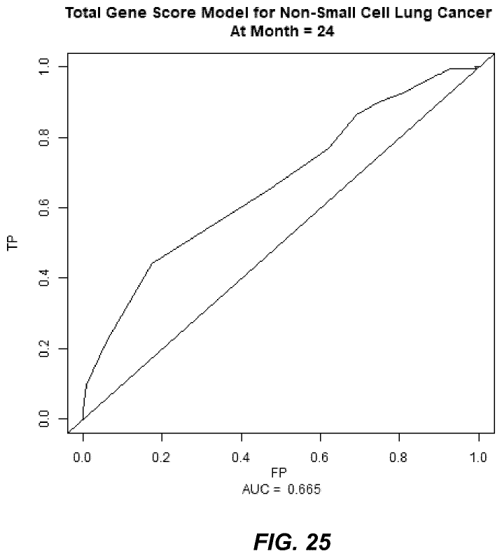

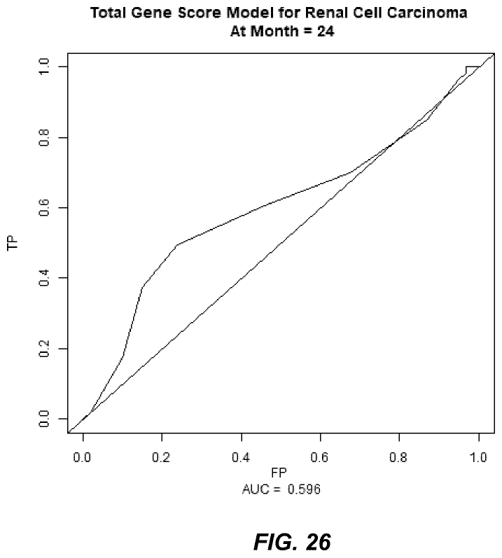

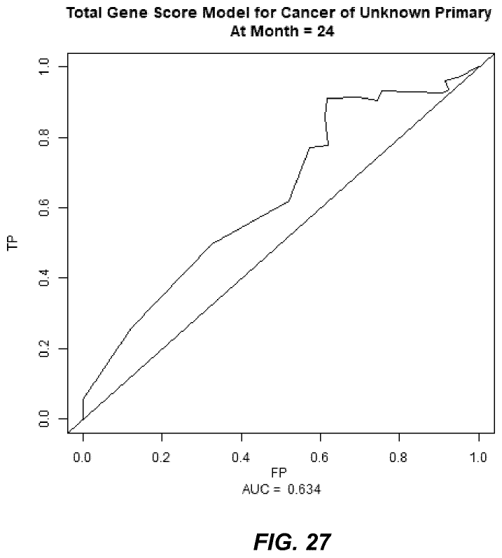

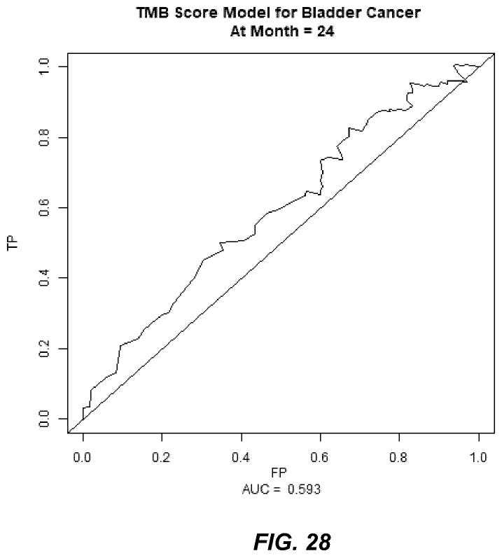

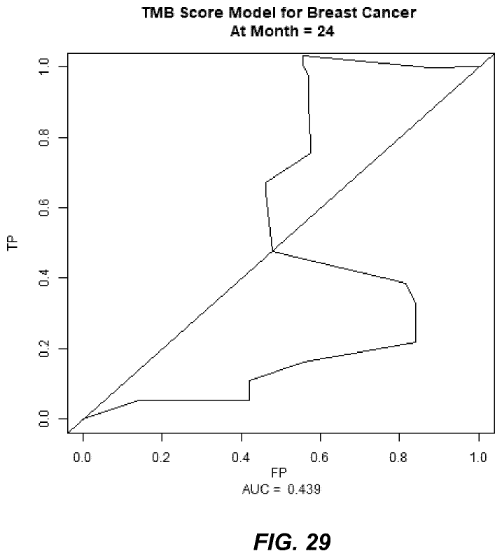

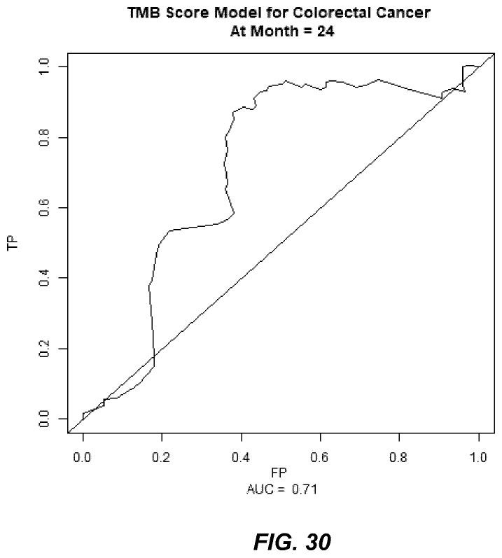

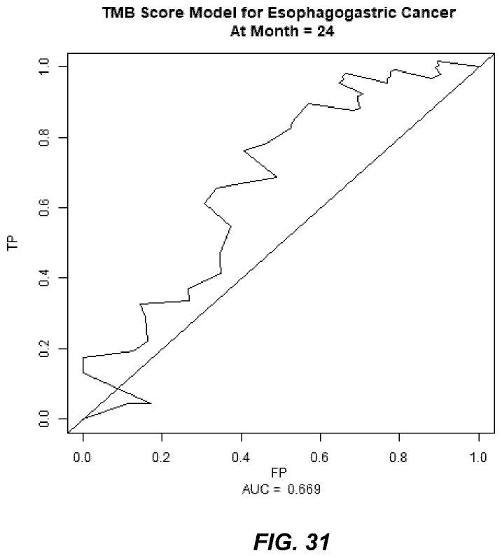

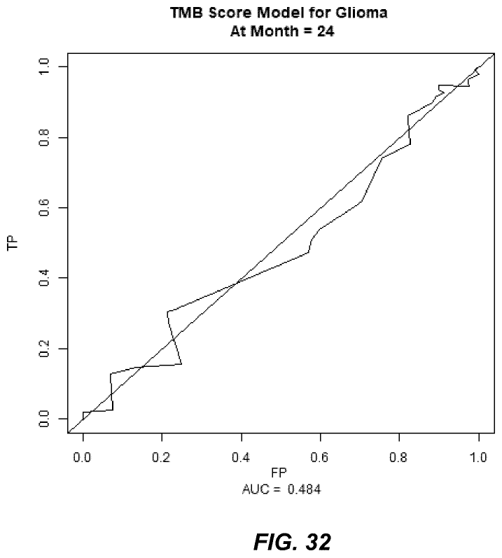

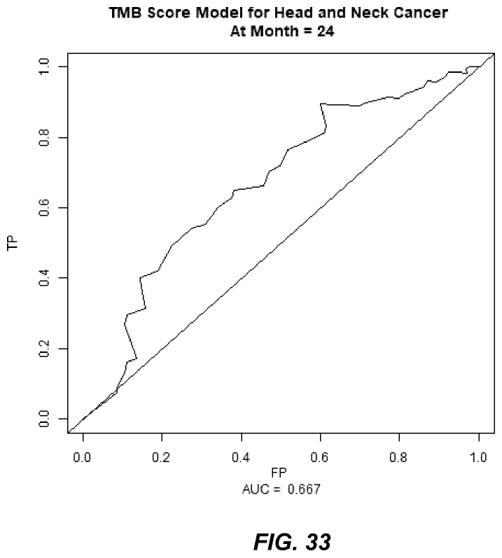

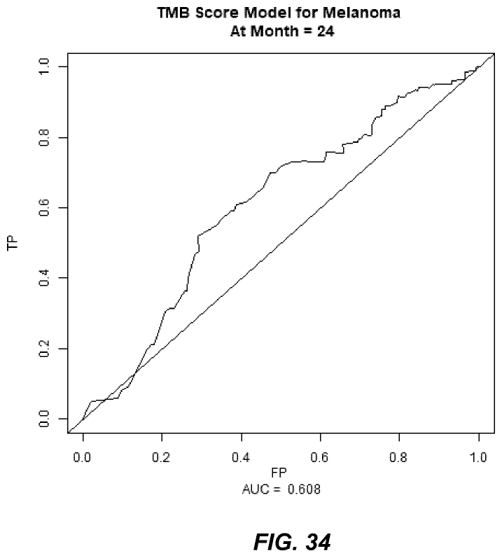

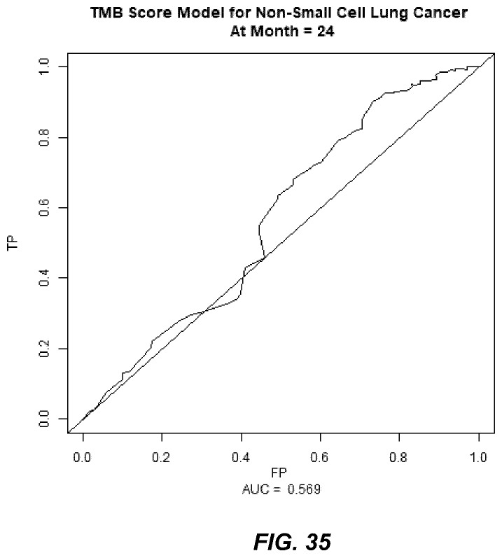

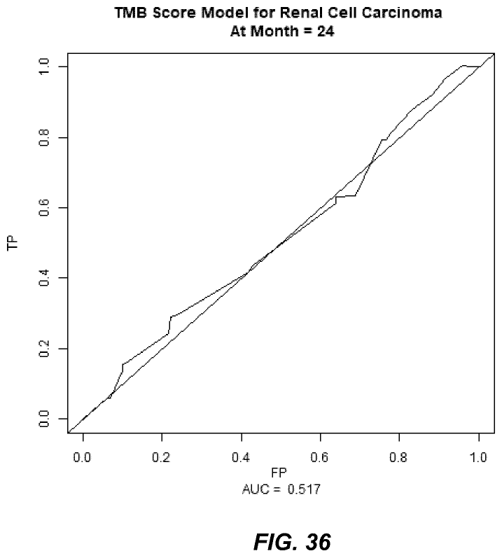

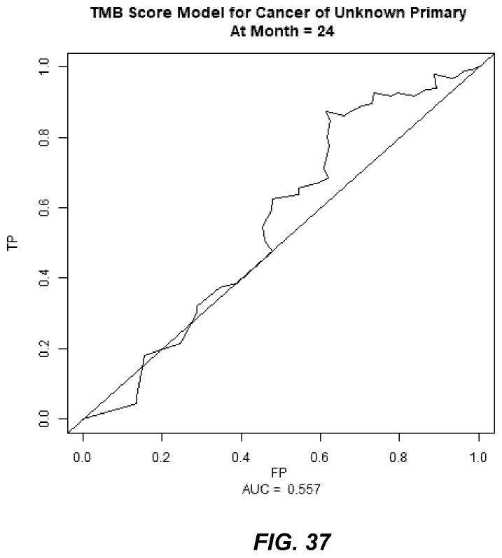

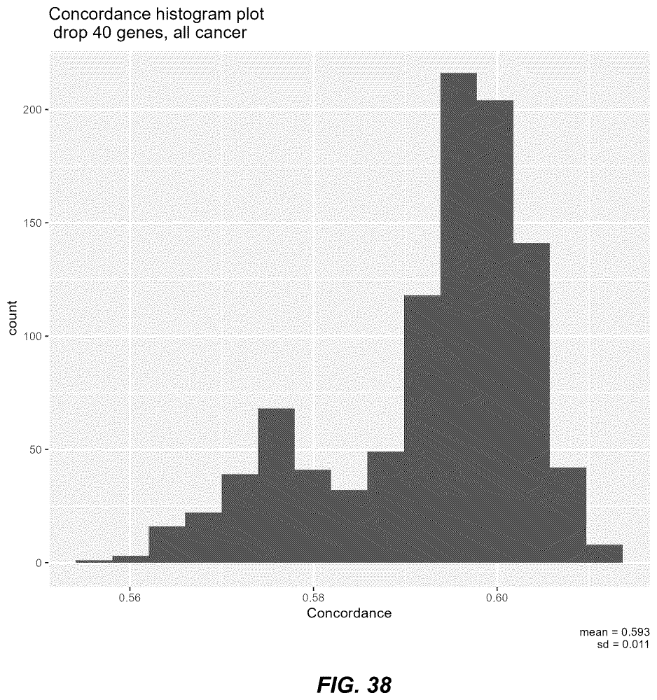

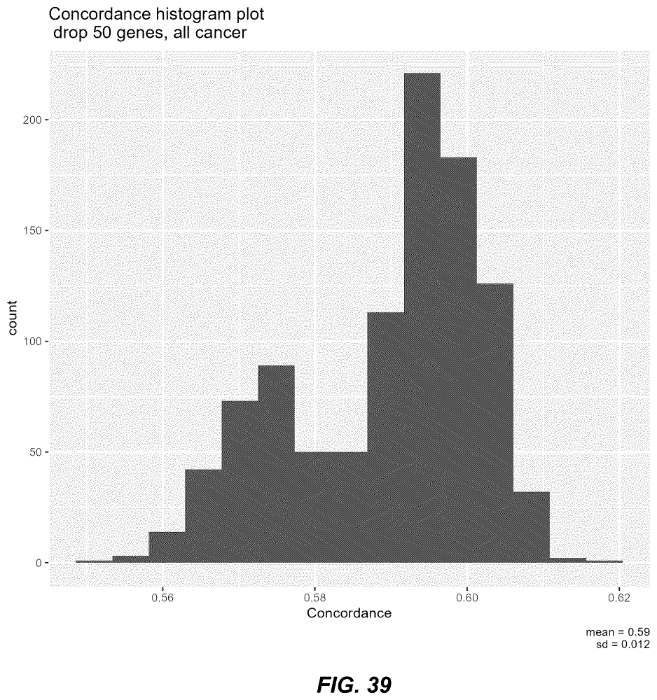

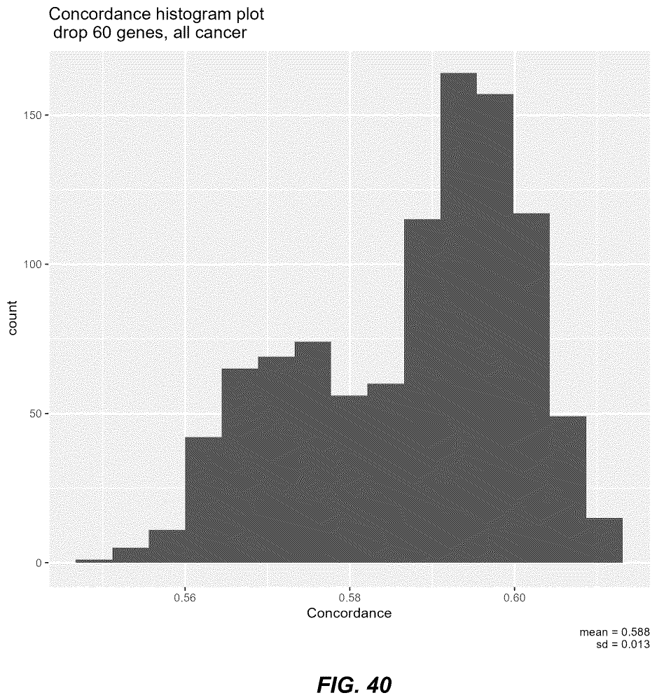

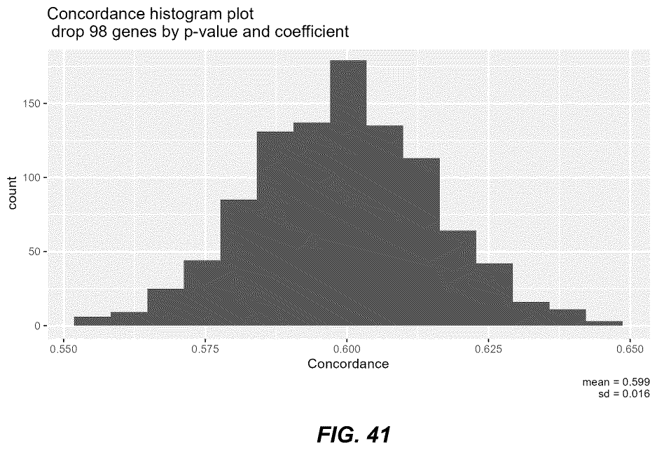

Among patients treated with chemotherapy alone, GS-O≥2 was not associated with survival improvement (p=0.33) indicating that it is a predictive marker and not merely prognostic (FIG. 2).

### DISCUSSION

Robust, clinically applicable predictors of response to ICI are currently lacking. The most commonly used and well-studied biomarkers, PD-L1 and TMB have significant limitations with respect to predictive ability when applied in a pan-cancer fashion due to the need for different cut-offs thereby undercutting their applicability. A study that evaluated PD-L1 as a predictive biomarker based on all US Food and Drug Administration (FDA) drug approvals of ICI through April 2019 showed that PD-L1 was predictive in only 28.9% of cases, and was either not predictive (53.3%) or not tested (17.8%) in the remaining cases (Davis A A, et al. J Immunother Cancer. 2019 7(1):278). There is no universal threshold for PD-L1 combined positive score (CPS) that can predict benefit. For example, pembrolizumab is approved in Head Neck squamous cell cancer (as a single agent), cervical Squamous cell cancer (as a single agent), gastric cancer (in combination with chemotherapy) for a CPS score >=1 while in esophageal squamous cell cancer (as a single agent) and triple negative breast cancer (in combination with chemotherapy), the CPS cut-off is >=10.

High tumor mutation burden (TMB-H) is another predictive biomarker for ICI response. The hypothesis is that a greater number of somatic mutations will lead to a larger number of potential neo-antigenic peptides thereby activating a protective immune response. The approval of the use of a TMB cut-off of >=10 mutations/Mb is based on a subset analysis of only 13% of patients who were enrolled on the KEYNOTE-158 trial which reported an ORR of 29% among the 102 subjects who exceeded this cut-off (Marabelle A, et al. The Lancet Oncology. 2020 21 (10): 1353-65).

However, a single threshold for TMB-High does not have universal predictive value and is limited by cancer-type. A study of more than 1660 ICI treated patients showed that ORR in the TMB-H patients exceeded TMB-Low patients in only 11 of 16 cancer types. Also, only patients in the top 20th percentile of TMB in each tumor type derived a survival benefit with ICI (Valero C, et al. JAMA Oncol. 2021 7 (5): 739-43; Samstein R M, et al. Nature Genetics. 2019 51 (2): 202-6). This strongly indicates that other cancer-specific mechanisms are at play. Specifically, a high somatic TMB does not predict for an intact antigen presentation machinery or a favorable tumor microenvironment that allows for T cell expansion. In cancer types in which neo-antigen load does not co-relate with CD8 T-cell levels such as breast cancer, prostate cancer, and glioma, TMB-H patients actually had a significantly lower ORR (15.3% vs. 23.4%) and worsened OS relative to TMB-L tumors (McGrail D J, et al. Annals of Oncology. 2021 32 (5): 661-72). Some TMB-H tumors have been shown to to limit antigen presentation by down-regulating the HLA/MHC axis which weakens TMB's applicability as a universal predictor of an immune response (Montesion M, et al. Cancer Discovery. 2021 11 (2): 282-92; Goodman A M, et al. Genome Med. 2020 12 (1): 45).

Somatic tumor genomic correlates of immune activation and evasion have been widely described in the literature but have as yet not been integrated into a comprehensive framework that addresses tumor and T cell-intrinsic mechanisms of activation and resistance. This paper addressed this gap by identifying putative genomic alterations that are positively and negatively associated with a response to ICI. We propose a holistic conceptual framework that goes beyond TMB and addresses the tumor biology that encompasses multiple pathways underlying DNA repair, TME, immune activation and resistance. The interplay of these dialectically opposed genomic factors was highly predictive of survival outcomes beyond TMB.

The study has several limitations; the retrospective nature of the findings in a single tumor type being the main one. External validation of these findings in other cohorts of patients treated with ICI is required. We aimed to develop a simple and widely applicable tool to assess genomic information that is predictive of survival with immunotherapy without the need for sophisticated bio-informatics based approaches for variant-calling or functional assessments of gene ontology. Many alterations selected in this scoring system are currently classified as “variants of unknown significance” though a deeper understanding of the functional significance of these alterations may become available in the future. Finally, we have identified more than 70 alterations across 24 functional domains that have an effect on survival after ICI. But, this list is not meant to be exhaustive and future studies will undoubtedly reveal more relevant gene alterations in other domains further refining and adding predictive power to this approach. Specifically, future iterations will need to incorporate the role of driver mutations that are commonly seen in tumors such as NSCLC but is rare in NENs.

A dialectic approach to critically analyzing the functional implications of genomic alterations can select patients with improved survival with ICI treatment. This has broad implications for future strategies to improve responses to immunotherapy. If somatic mutations dictate to a large extent the immunogenicity of tumors, future research efforts should focus on abrogating adverse tumor-specific genomic pathways of immune evasion and resistance. Without incorporating mitigating strategies, merely inhibiting other immune checkpoints such as IDO, ICOS, TIGIT, TIM-3 are unlikely to augment the immune response, a potential explanation for the failure of multiple large recent clinical trials that sought to inhibit novel immune checkpoints.

### Conclusion

Reported here is the largest cohort hitherto of NENs treated with immunotherapy showing that 28% of patients derive sustained benefit lasting a year or more. A genomic score was derived that was predictive of long-term survival benefit in patients treated with immunotherapy. The applicability of this score as a pan-cancer biomarker is expected to work for other cancer types.

### Example 2: Derivation of a Genomic Score that Predicts Response to Immune Checkpoint Blockade

### Background

We conducted an institutional review of patients with neuroendocrine neoplasms (NENs) treated with anti-PD-1 and anti-cytotoxic T-lymphocyte-associated protein 4 (CTLA-4) antibodies to better understand real world outcomes and toxicity in NENs.

### Methods

Retrospective, cohort study of clinico-genomic data was performed. Patients who have received at least one dose of ipilimumab and/or nivolumab between Sep. 1, 2017 and Mar. 31, 2023 were included.

We derived a genomic score (GS) based on alterations in a panel of genes that are well-described predictors of response to immunotherapy. These were categorized and scored as in Table 1. We assessed the relationship of GS with overall survival (OS).

### Results

A total of 48 patients met inclusion criteria. Median age was 59 (range 27-80). The primary site was hindgut (10%), pancreas (35%), lung (8%), small bowel/unknown (20%) and esophageal/gastric (8%). Most patients (85%) had grade 3 NEN, 10% grade, and 4% unknown Morphology was well-differentiated in 38% and poorly differentiated in 60%. The cohort was heavily pre-treated; 46% had progression on ≥3 prior lines of therapy. PD-L1 testing was positive in 7% ( 2/28) of patients with available samples. Median OS was 5.9 months (95% CI, 4.5-7.4], and 27% of patients survived for a year or more. ≥Grade 3 adverse events were seen in 17% and systemic steroids were administered in 27% of patients.

Complete somatic NGS testing information was available in 81% ( 39/48) of patients and the association of GS with OS was studied in this population. The median OS was not reached in patients with GS≥2 vs. 5 months in those with GS<2 (HR 0.01, p<0.001). Among patients with OS≥11 months, 85% ( 11/13) had a GS≥2, whereas among patients who died before 11 months, 9% ( 2/23) had a GS>=2 (Fisher's Exact test p<0.001). After adjusting for age, primary site, Ki-67% and differentiation, GS>=2 was independently, significantly associated with OS (HR=0.03, p<0.001).

### Conclusion

We report the largest cohort hitherto of NENs treated with immunotherapy and find that 27% of patients derive sustained benefit. We derived a genomic score that was associated with survival which will need prospective validation.

### Example 3: Additional Data

Univariate Cox proportional hazards regression models were fitted using total score and tumor mutation burden (TMB) for all patients and within cancer types. The C-index reflects the predictive accuracy of the Cox regression model. Concordance indices of Cox regression models. The “Cancer” column indicates whether the model was fitted for all patients or for a specific cancer type. The “Concordance” column reports the concordance index (C-index) of each model, while the “CI” column provides the corresponding 95% confidence interval. The column “Score Used” specifies whether the model was based on total gene score or TMB.

TABLE 2

| | Concor- | | |
|---|---|---|---|
| Cancer | dance | CI | Score Used |
| all | 0.602 | (0.581, 0.623) | Total Gene Score |
| all | 0.544 | (0.523, 0.565) | TMB Score |
| Bladder Cancer | 0.559 | (0.493, 0.624) | Total Gene Score |
| Bladder Cancer | 0.563 | (0.499, 0.627) | TMB Score |
| Breast Cancer | 0.589 | (0.47, 0.708) | Total Gene Score |
| Breast Cancer | 0.458 | (0.33, 0.586) | TMB Score |
| Cancer of Unknown | 0.517 | (0.417, 0.616) | Total Gene Score |
| Primary | | | |
| Cancer of Unknown | 0.514 | (0.406, 0.621) | TMB Score |
| Primary | | | |
| Colorectal Cancer | 0.549 | (0.448, 0.651) | Total Gene Score |
| Colorectal Cancer | 0.557 | (0.456, 0.659) | TMB Score |
| Esophagogastric Cancer | 0.547 | (0.458, 0.635) | Total Gene Score |
| Esophagogastric Cancer | 0.544 | (0.456, 0.631) | TMB Score |
| Glioma | 0.446 | (0.374, 0.518) | Total Gene Score |
| Glioma | 0.452 | (0.381, 0.524) | TMB Score |
| Head and Neck Cancer | 0.59 | (0.522, 0.658) | Total Gene Score |
| Head and Neck Cancer | 0.573 | (0.5, 0.646) | TMB Score |
| Melanoma | 0.582 | (0.529, 0.635) | Total Gene Score |
| Melanoma | 0.576 | (0.524, 0.628) | TMB Score |
| Non-Small Cell Lung | 0.604 | (0.565, 0.643) | Total Gene Score |
| Cancer | | | |
| Non-Small Cell Lung | 0.536 | (0.494, 0.578) | TMB Score |
| Cancer | | | |
| Renal Cell Carcinoma | 0.614 | (0.528, 0.701) | Total Gene Score |
| Renal Cell Carcinoma | 0.542 | (0.461, 0.623) | TMB Score |

Univariate Cox regression model p-values, absolute values of the model coefficients as well as their corresponding rankings for each gene. The “Gene” column lists the gene names. The “P-value” column reports the Cox regression model p-value, while the “Absolute Value of Coefficient” column provides the magnitude of the model coefficients. The column “P-value Rank” shows the ranking of genes based on p-values, from largest to smallest. The column “Absolute Value of Coefficient Rank” shows the ranking of the genes based on the absolute value of coefficients, from smallest to largest. The “Final Rank” column represents the average of the two rankings, used to determine the overall importance of each gene. The 12 genes that were not represented in any patients were listed at the bottom of the table.

TABLE 3

| | | | | | Absolute | |
|---|---|---|---|---|---|---|
| | | | Absolute | | Value of | |
| Functional | | | Value of | P-value | Coefficient | Final |
| Category | Gene | P-value | Coefficient | Rank | Rank | Rank |
| RNA Polymerase II | CDK8 | 0.992 | 0 | 1 | 1 | 1 |
| Transcription and | | | | | | |
| Translation | | | | | | |
| Mismatch Repair | PMS1 | 0.923 | 0.03 | 3 | 3 | 3 |
| Inhibition of Tumor | EGFR | 0.876 | 0.02 | 6 | 2 | 4 |
| Growth Signaling | | | | | | |
| Inhibition of Tumor | ESR1 | 0.885 | 0.03 | 4 | 4 | 4 |
| Growth Signaling | | | | | | |
| Inhibition of Tumor | REL | 0.877 | 0.05 | 5 | 6 | 5.5 |
| Growth Signaling | | | | | | |
| Increased Anti-oxidant | NFE2L2 | 0.862 | 0.04 | 8 | 5 | 6.5 |
| Signaling | | | | | | |
| Linker Histone | HIST1H1C | 0.856 | 0.06 | 9 | 10 | 9.5 |
| Loss of MHC | SH2D1A** | 0.866 | 0.07 | 7 | 13 | 10 |
| Presentation | | | | | | |
| Inhibition of Tumor | FGFR3 | 0.783 | 0.05 | 13 | 8 | 10.5 |
| Growth Signaling | | | | | | |
| Increased mTOR | PTEN | 0.779 | 0.05 | 14 | 7 | 10.5 |
| Signaling and Reduced | Damaging | | | | | |
| Mitophagy | Mutation | | | | | |
| RNA Polymerase II | EIF4A2 | 0.841 | 0.08 | 10 | 14 | 12 |
| Transcription and | | | | | | |
| Translation | | | | | | |
| SWI/SNF Sub-units | SMARCB1 | 0.795 | 0.09 | 12 | 15 | 13.5 |
| Loss of Negative | SOCS1 | 0.823 | 0.09 | 11 | 16 | 13.5 |
| Regulators of Cytokine | | | | | | |
| Signaling | | | | | | |
| Loss of Degradation of | FBXW7 | 0.733 | 0.06 | 15 | 12 | 13.5 |
| NOTCH1/2 | | | | | | |
| SWI/SNF Sub-units | SMARCA4 | 0.64 | 0.06 | 20 | 11 | 15.5 |
| Increase in Myeloid Cell | DNMT3B | 0.703 | 0.09 | 17 | 17 | 17 |
| Population | | | | | | |
| Activators of Growth | KRAS | 0.574 | 0.06 | 26 | 9 | 17.5 |
| Signaling | | | | | | |
| Mismatch Repair | MLH1 | 0.681 | 0.12 | 18 | 21 | 19.5 |
| Inhibition of Tumor | RASA1 | 0.637 | 0.12 | 21 | 20 | 20.5 |
| Growth Signaling | | | | | | |
| Increase in Myeloid Cell | WT1 | 0.664 | 0.12 | 19 | 22 | 20.5 |
| Population | | | | | | |
| RNA Polymerase II | DROSHA | 0.597 | 0.11 | 23 | 19 | 21 |
| Transcription and | | | | | | |
| Translation | | | | | | |
| Increased TGF-Beta | SMAD4 | 0.591 | 0.1 | 24 | 18 | 21 |
| Signaling | | | | | | |
| Increased MYC | MYC | 0.707 | 0.14 | 16 | 26 | 21 |
| Signaling | | | | | | |
| Loss of MHC | B2M | 0.575 | 0.14 | 25 | 25 | 25 |
| Presentation | | | | | | |
| Loss of TGFB Signaling | TGFBR2 | 0.526 | 0.14 | 28 | 24 | 26 |
| Inhibition of Tumor | INPP4B | 0.516 | 0.17 | 30 | 29 | 29.5 |
| Growth Signaling | | | | | | |
| RNA processing and | DROSHA | 0.597 | 0.22 | 22 | 40 | 31 |
| Export | | | | | | |
| RNA processing and | XPO1 | 0.502 | 0.2 | 31 | 33 | 32 |
| Export | | | | | | |
| Mismatch Repair | PMS2 | 0.522 | 0.2 | 29 | 35 | 32 |
| RNA Polymerase II | XPO1 | 0.502 | 0.2 | 32 | 34 | 33 |
| Transcription and | | | | | | |
| Translation | | | | | | |
| Increased WNT | APC | 0.324 | 0.12 | 47 | 23 | 35 |
| Signaling | | | | | | |
| Loss of MHC | HLA-A | 0.494 | 0.22 | 34 | 42 | 38 |
| Presentation | | | | | | |
| Increased MYC | MYCN | 0.47 | 0.22 | 35 | 43 | 39 |
| Signaling | | | | | | |
| Increased WNT | CTNNB1 | 0.313 | 0.19 | 49 | 31 | 40 |
| Signaling | | | | | | |
| RNA Polymerase II | CDK12 | 0.341 | 0.21 | 46 | 37 | 41.5 |
| Transcription and | | | | | | |
| Translation | | | | | | |
| Loss of MHC | TRAF7 | 0.41 | 0.24 | 39 | 45 | 42 |
| Presentation | | | | | | |
| Mutations in NOTCH | NOTCH1 | 0.239 | 0.18 | 55 | 30 | 42.5 |
| Pathway | | | | | | |
| DNA Polymerases | POLD1 | 0.207 | 0.16 | 60 | 27 | 43.5 |
| DNA Damage Repair | BLM | 0.367 | 0.23 | 44 | 44 | 44 |
| RNA Polymerase II | TBX3 | 0.294 | 0.22 | 51 | 41 | 46 |
| Transcription and | | | | | | |
| Translation | | | | | | |
| DNA Damage Repair | CHEK2 | 0.387 | 0.26 | 42 | 51 | 46.5 |
| Chromatin Remodeling/ | EP300 | 0.219 | 0.21 | 58 | 36 | 47 |
| Epigenetic Functions | | | | | | |
| Regulation of | RARA | 0.409 | 0.29 | 40 | 56 | 48 |
| Transcription | | | | | | |
| Activators of Growth | PIK3R3 | 0.441 | 0.32 | 37 | 60 | 48.5 |
| Signaling | | | | | | |
| Immune Escape | PDCD1 | 0.386 | 0.29 | 43 | 55 | 49 |
| RNA Polymerase II | EIF1AX | 0.402 | 0.3 | 41 | 58 | 49.5 |
| Transcription and | | | | | | |
| Translation | | | | | | |
| Inhibition of Tumor | IRS1 | 0.288 | 0.25 | 52 | 48 | 50 |
| Growth Signaling | | | | | | |
| Chromatin Remodeling/ | BRD4 | 0.258 | 0.26 | 54 | 50 | 52 |
| Epigenetic Functions | | | | | | |
| DNA Damage Repair | CHEK1 | 0.459 | 0.37 | 36 | 68 | 52 |
| Dysregulated RNA | RBM10 | 0.16 | 0.21 | 69 | 38 | 53.5 |
| Splicing | | | | | | |
| Chromatin Remodeling/ | CARM1 | 0.533 | 0.44 | 27 | 81 | 54 |
| Epigenetic Functions | | | | | | |
| Histone Post- | BAP1 | 0.197 | 0.25 | 64 | 47 | 55.5 |
| translational | | | | | | |
| Modification | | | | | | |
| Increased WNT | AXIN1 | 0.227 | 0.3 | 57 | 57 | 57 |
| Signaling | | | | | | |
| Reduced Cytokine | JAK1 | 0.231 | 0.31 | 56 | 59 | 57.5 |
| Signaling | | | | | | |
| Chromatin Remodeling/ | DOT1L | 0.056 | 0.17 | 90 | 28 | 59 |
| Epigenetic Functions | | | | | | |
| Inhibition of Tumor | NTRK1 | 0.18 | 0.27 | 66 | 52 | 59 |
| Growth Signaling | | | | | | |
| Inhibition of Tumor | HRAS | 0.272 | 0.37 | 53 | 66 | 59.5 |
| Growth Signaling | | | | | | |
| Reduced Checkpoint | CD274 | 0.412 | 0.47 | 38 | 86 | 62 |
| Expression | | | | | | |
| DNA Damage Repair | MUTYH | 0.302 | 0.42 | 50 | 78 | 64 |
| Loss of TGFB Signaling | BMPR1A | 0.318 | 0.45 | 48 | 82 | 65 |
| Increased WNT | AMER1 | 0.161 | 0.33 | 68 | 63 | 65.5 |
| Signaling | | | | | | |
| DNA Damage | ATR | 0.038 | 0.2 | 102 | 32 | 67 |
| Transducers | | | | | | |
| Chromatin Remodeling/ | ATRX | 0.068 | 0.26 | 85 | 49 | 67 |
| Epigenetic Functions | | | | | | |
| Increased WNT | AXIN2 | 0.148 | 0.37 | 72 | 65 | 68.5 |
| Signaling | | | | | | |
| Inhibition of Tumor | ETV6 | 0.207 | 0.42 | 61 | 77 | 69 |
| Vascularization | | | | | | |
| DNA Damage Repair | RECQL | 0.348 | 0.54 | 45 | 97 | 71 |
| Spliceosome and | SF3B1 | 0.082 | 0.33 | 81 | 62 | 71.5 |
| Reduced Non-sense | | | | | | |
| Mediated Decay | | | | | | |
| Loss of TGFB Signaling | TGFBR1 | 0.15 | 0.42 | 71 | 75 | 73 |
| Inhibition of Tumor | ETV1 | 0.199 | 0.46 | 63 | 84 | 73.5 |
| Growth Signaling | | | | | | |
| Chromatin Remodeling/ | KMT2D | 0.027 | 0.24 | 107 | 46 | 76.5 |
| Epigenetic Functions | | | | | | |
| DNA Damage Repair | BABAM1 | 0.496 | 0.68 | 33 | 126 | 79.5 |
| Immune Escape | SH2D1A* | 0.986 | 7 | 2 | 159 | 80.5 |
| DNA Damage | ATM | 0.009 | 0.22 | 123 | 39 | 81 |
| Transducers | | | | | | |
| Inhibition of Tumor | MAP3K1 | 0.083 | 0.45 | 80 | 83 | 81.5 |
| Growth Signaling | | | | | | |
| Inhibition of Tumor | ERBB3 | 0.053 | 0.42 | 91 | 76 | 83.5 |
| Growth Signaling | | | | | | |
| Activators of Growth | NF1 | 0.017 | 0.28 | 115 | 53 | 84 |
| Signaling | | | | | | |
| Increase in Myeloid Cell | DNMT3A | 0.077 | 0.51 | 83 | 91 | 87 |
| Population | | | | | | |
| Spliceosome and | U2AF1 | 0.156 | 0.58 | 70 | 109 | 89.5 |
| Reduced Non-sense | | | | | | |
| Mediated Decay | | | | | | |
| Mutations in NOTCH | NOTCH4 | 0.019 | 0.37 | 113 | 67 | 90 |
| Pathway | | | | | | |
| Inhibition of Tumor | PDGFRA | 0.026 | 0.41 | 109 | 73 | 91 |
| Growth Signaling | | | | | | |
| Inhibition of Tumor | INSR | 0.044 | 0.48 | 96 | 87 | 91.5 |
| Growth Signaling | | | | | | |
| Increased Anti-oxidant | CUL3 | 0.072 | 0.55 | 84 | 99 | 91.5 |
| Signaling | | | | | | |
| DNA Damage Repair | FANCC | 0.165 | 0.62 | 67 | 117 | 92 |
| Inhibition of Tumor | AKT2 | 0.138 | 0.61 | 73 | 113 | 93 |
| Growth Signaling | | | | | | |
| DNA Damage Repair | BRCA2 | 0.015 | 0.4 | 116 | 72 | 94 |
| Inhibition of Tumor | ERBB4 | 0.014 | 0.38 | 118 | 70 | 94 |
| Growth Signaling | | | | | | |
| Inhibition of Tumor | DDR2 | 0.059 | 0.55 | 88 | 101 | 94.5 |
| Growth Signaling | | | | | | |
| Mismatch Repair | MSH3 | 0.218 | 0.71 | 59 | 131 | 95 |
| Mismatch Repair | MSH6 | 0.043 | 0.53 | 97 | 94 | 95.5 |
| Loss of Negative | PTPRS | 0.02 | 0.43 | 112 | 79 | 95.5 |
| Regulators of Cytokine | | | | | | |
| Signaling | | | | | | |
| Inhibition of Tumor | IRF4 | 0.102 | 0.62 | 77 | 116 | 96.5 |
| Growth Signaling | | | | | | |
| Microtubule Assembly | AURKA | 0.113 | 0.65 | 75 | 121 | 98 |
| SWI/SNF Sub-units | PBRM1 | 0.006 | 0.37 | 127 | 69 | 98 |
| DNA Polymerases | POLE | 0.002 | 0.29 | 143 | 54 | 98.5 |
| SWI/SNF Sub-units | ARID1A | 0.005 | 0.35 | 133 | 64 | 98.5 |
| DNA Damage Repair | FANCA | 0.046 | 0.56 | 93 | 104 | 98.5 |
| Regulation of | BCL2 | 0.203 | 0.74 | 62 | 135 | 98.5 |
| Transcription | | | | | | |
| DNA Damage Repair | AURKA | 0.113 | 0.65 | 76 | 122 | 99 |
| Activators of Growth | AKT1 | 0.094 | 0.64 | 78 | 120 | 99 |
| Signaling | | | | | | |
| SWI/SNF Sub-units | ARID2 | 0.007 | 0.42 | 125 | 74 | 99.5 |
| Inhibition of Tumor | HGF | 0.02 | 0.49 | 111 | 88 | 99.5 |
| Growth Signaling | | | | | | |
| Mismatch Repair | MSH2 | 0.045 | 0.56 | 95 | 105 | 100 |
| Inhibition of Tumor | MST1R | 0.04 | 0.55 | 98 | 102 | 100 |
| Growth Signaling | | | | | | |
| Increased Anti-oxidant | KEAP1 | 0.006 | 0.38 | 129 | 71 | 100 |
| Signaling | | | | | | |
| SWI/SNF Sub-units | SMARCD1 | 0.127 | 0.68 | 74 | 127 | 100.5 |
| Inhibition of Tumor | PIK3C3 | 0.04 | 0.55 | 99 | 103 | 101 |
| Growth Signaling | | | | | | |
| Chromatin Remodeling/ | KMT2B | 0.06 | 0.63 | 87 | 119 | 103 |
| Epigenetic Functions | | | | | | |
| Inhibition of Tumor | PIK3CG | 0.009 | 0.47 | 122 | 85 | 103.5 |
| Growth Signaling | | | | | | |
| DNA Damage Repair | BRIP1 | 0.038 | 0.56 | 103 | 106 | 104.5 |
| Regulation of | BCOR | 0.014 | 0.53 | 117 | 95 | 106 |
| Transcription | | | | | | |
| Increased mTOR | RICTOR | 0.031 | 0.58 | 105 | 108 | 106.5 |
| Signaling and Reduced | | | | | | |
| Mitophagy | | | | | | |
| Loss of MHC | TNFRSF14 | 0.088 | 0.77 | 79 | 137 | 108 |
| Presentation | | | | | | |
| SWI/SNF Sub-units | ARID1B | 0.007 | 0.52 | 124 | 93 | 108.5 |
| DNA Damage Repair | ERCC5 | 0.04 | 0.62 | 100 | 118 | 109 |
| Loss of TGFB Signaling | INHBA | 0.033 | 0.62 | 104 | 114 | 109 |
| Activators of Growth | NFKBIA | 0.182 | 1.34 | 65 | 154 | 109.5 |
| Signaling | | | | | | |
| Inhibition of MYC | MGA | 0.006 | 0.52 | 128 | 92 | 110 |
| Signaling | | | | | | |
| Inhibition of Tumor | KDR | 0.005 | 0.5 | 131 | 89 | 110 |
| Vascularization | | | | | | |
| Inhibition of Tumor | VHL | <0.001 | 0.32 | 159 | 61 | 110 |
| Vascularization | | | | | | |
| Inhibition of Tumor | RPS6KB2 | 0.057 | 0.72 | 89 | 133 | 111 |
| Growth Signaling | | | | | | |
| Chromatin Remodeling/ | KMT2C | 0.001 | 0.44 | 146 | 80 | 113 |
| Epigenetic Functions | | | | | | |
| Inhibition of Tumor | FLT4 | 0.006 | 0.55 | 126 | 100 | 113 |
| Vascularization | | | | | | |
| Inhibition of Tumor | FGFR4 | 0.039 | 0.69 | 101 | 129 | 115 |
| Growth Signaling | | | | | | |
| Inhibition of Tumor | PDGFRB | 0.009 | 0.6 | 121 | 112 | 116.5 |
| Growth Signaling | | | | | | |
| Increased WNT | TCF7L2 | 0.022 | 0.66 | 110 | 123 | 116.5 |
| Signaling | | | | | | |
| Inhibition of MYC | MAX | 0.078 | 1.76 | 82 | 157 | 119.5 |
| Signaling | | | | | | |
| RNA Polymerase II | CDK6 | 0.063 | 1.31 | 86 | 153 | 119.5 |
| Transcription and | | | | | | |
| Translation | | | | | | |
| Chromatin Remodeling/ | KMT2A | 0.001 | 0.54 | 145 | 96 | 120.5 |
| Epigenetic Functions | | | | | | |
| Loss of Negative | PTPRT | <0.001 | 0.51 | 154 | 90 | 122 |
| Regulators of Cytokine | | | | | | |
| Signaling | | | | | | |
| DNA Damage Repair | MRE11A | 0.029 | 0.83 | 106 | 140 | 123 |
| DNA Damage Repair | NBN | 0.026 | 0.79 | 108 | 139 | 123.5 |
| Inhibition of Tumor | MST1 | 0.046 | 1.41 | 94 | 155 | 124.5 |
| Growth Signaling | | | | | | |
| Inhibition of Tumor | PIK3C2G | 0.002 | 0.57 | 142 | 107 | 124.5 |
| Growth Signaling | | | | | | |
| Mutations in NOTCH | NOTCH2 | 0.002 | 0.58 | 140 | 110 | 125 |
| Pathway | | | | | | |
| Increased MYC | MYCL | 0.051 | 1.95 | 92 | 158 | 125 |
| Signaling | | | | | | |
| Increase in Myeloid Cell | TET2 | 0.003 | 0.59 | 139 | 111 | 125 |
| Population | | | | | | |
| Increased mTOR | STK11 | <0.001 | 0.54 | 155 | 98 | 126.5 |
| Signaling and Reduced | | | | | | |
| Mitophagy | | | | | | |
| Increase in Myeloid Cell | ASXL1 | 0.005 | 0.67 | 132 | 124 | 128 |
| Population | | | | | | |
| Reduced Cytokine | JAK2 | 0.013 | 0.84 | 120 | 141 | 130.5 |
| Signaling | | | | | | |
| RNA Polymerase II | MED12 | 0.003 | 0.68 | 138 | 125 | 131.5 |
| Transcription and | | | | | | |
| Translation | | | | | | |
| Regulation of | BCL6 | 0.017 | 1.19 | 114 | 152 | 133 |
| Transcription | | | | | | |
| DNA Damage Repair | RAD50 | 0.013 | 0.95 | 119 | 149 | 134 |
| Sonic Hedgehog | SMO | 0.005 | 0.85 | 130 | 142 | 136 |
| Signaling | | | | | | |
| Loss of Negative | PTPRD | <0.001 | 0.62 | 157 | 115 | 136 |
| Regulators of Cytokine | | | | | | |
| Signaling | | | | | | |
| Sonic Hedgehog | PTCH1 | 0.003 | 0.77 | 135 | 138 | 136.5 |
| Signaling | | | | | | |
| Inhibition of Tumor | FLT3 | 0.002 | 0.76 | 144 | 136 | 140 |
| Growth Signaling | | | | | | |
| Immune Escape | ZFHX3 | <0.001 | 0.69 | 153 | 128 | 140.5 |
| Loss of Myeloid Cell | CSF1R | 0.003 | 0.89 | 136 | 146 | 141 |
| population | | | | | | |
| Mutations in NOTCH | NOTCH3 | <0.001 | 0.71 | 151 | 132 | 141.5 |
| Pathway | | | | | | |
| Immune Escape | CSF1R | 0.003 | 0.89 | 137 | 147 | 142 |
| Increase in Myeloid Cell | DNMT1 | 0.002 | 0.89 | 141 | 145 | 143 |
| Population | | | | | | |
| Chromatin Remodeling/ | SETD2 | <0.001 | 0.71 | 158 | 130 | 144 |
| Epigenetic Functions | | | | | | |
| Loss of Negative | PPM1D | 0.004 | 1.65 | 134 | 156 | 145 |
| Regulators of Cytokine | | | | | | |
| Signaling | | | | | | |
| Loss of TGFB Signaling | PAK7 | <0.001 | 0.72 | 156 | 134 | 145 |
| Loss of MHC | IL7R | <0.001 | 0.87 | 149 | 143 | 146 |
| Presentation | | | | | | |
| Inhibition of Tumor | NTRK3 | <0.001 | 0.87 | 152 | 144 | 148 |
| Growth Signaling | | | | | | |
| Inhibition of Tumor | RET | <0.001 | 0.94 | 148 | 148 | 148 |
| Growth Signaling | | | | | | |
| Increased YAP | LATS1 | <0.001 | 1.01 | 147 | 150 | 148.5 |
| Signaling | | | | | | |
| Increased WNT | RNF43 | <0.001 | 1.03 | 150 | 151 | 150.5 |
| Signaling | | | | | | |
| RNA processing and | DICER | / | / | / | / | / |
| Export | | | | | | |
| DNA Damage Repair | FANCB | / | / | / | / | / |
| DNA Damage Repair | FANCD2 | / | / | / | / | / |
| DNA Damage Repair | FANCL | / | / | / | / | / |
| Loss of Negative | PTPRO | / | / | / | / | / |
| Regulators of Cytokine | | | | | | |
| Signaling | | | | | | |
| RNA Polymerase II | DICER | / | / | / | / | / |
| Transcription and | | | | | | |
| Translation | | | | | | |
| Linker Histone | HIST1H1E | / | / | / | / | / |
| Increased YAP | AJUBA | / | / | / | / | / |
| Signaling | | | | | | |
| Increased YAP | JUB | / | / | / | / | / |
| Signaling | | | | | | |
| Reduced Checkpoint | CD273 | / | / | / | / | / |
| Expression | | | | | | |
| Reduced NOTCH | NOTCH Loss | / | / | / | / | / |
| Pathway Signaling | | | | | | |
| Activators of Growth | ERRFI2 | / | / | / | / | / |
| Signaling | | | | | | |

*= frameshift or deletion
**= any alteration other than frameshift or deletion

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of treating a solid tumor in a subject comprising:
(a) gene sequencing a tumor sample from the subject to identify gene alterations compared to wildtype controls in at least 70 of the genes selected from the group consisting of AMER1, APC, ARID1A, ARID1B, ARID2, ASXL1, ATM, ATR, ATRX, AURKA, AXIN1, AXIN2, B2M, BABAM1, BAP1, BCL2, BCL6, BCOR, BLM, BMPR1A, BRCA2, BRD4, BRIP1, CARM1, CD273, CD274, CDK12, CDK6, CDK8, CHEK1, CHEK2, CSF1R, CTNNB1, CUL3, DDR2, DICER, DNMT1, DNMT3A, DNMT3B, DOT1L, DROSHA, EGFR, EIF1AX, EIF4A2, EP300, ERBB3, ERBB4, ERCC5, ERRF12, ESR1, ETV1, ETV6, FANCA, FANCB, FANCC, FANCD2, FANCL, FBXW7, FGFR3, FGFR4, FLT3, FLT4, HGF, HIST1H1C, HIST1H1E, HLA-A, HRAS, IL7R, INHBA, INPP4B, INSR, IRF4, IRS1, JAK1, JAK2, JUB, KDR, KEAP1, KMT2A, KMT2B, KMT2C, KMT2D, KRAS, LATS1, MAP3K1, MAX, MED12, MGA, MLH1, MRE11A, MSH2, MSH3, MSH6, MST1, MST1R, MUTYH, MYC, MYCL, MYCN, NBN, NF1, NFE2L2, NFKBIA, NOTCH, NOTCH1, NOTCH2, NOTCH3, NOTCH4, NTRK1, NTRK3, PAK7, PBRM1, PDCD1, PDGFRA, PDGFRB, PIK3C2G, PIK3C3, PIK3CG, PIK3R3, PMS1, PMS2, POLD1, POLE, PPM1D, PTCH1, PTEN, PTPRD, PTPRO, PTPRS, PTPRT, RAD50, RARA, RASA1, RBM10, RECQL, REL, RET, RICTOR, RNF43, RPS6KB2, SETD2, SF3B1, SH2D1A, SMAD4, SMARCA4, SMARCB1, SMARCD1, SMO, SOCS1, STK11, TBX3, TCF7L2, TET2, TGFBR1, TGFBR2, TNFRSF14, TRAF7, U2AF1, VHL, WT1, XPO1, and ZFHX3;
(b) calculating a survival score from the gene alterations based on the following:
wherein the presence of an amino acid substitution in a gene selected from the group consisting of ATR, ATM, POLE, and POLD1 increases the survival score by 2(x) for each gene alteration,
wherein the presence of an amino acid substitution in a gene selected from the group consisting of ATRX, AJUBA, AKT2, ARID1A, ARID2, ARIDIB, AURKA, BABAM1, BCL2, BCL6, BCOR, BLM, BMPR1A, BRCA2, BRD4, BRIP1, CARM1, CDK12, CDK6, CDK8, CHEK1, CHEK2, CSF1R, DICER, DOT1L, DROSHA, EGFR, EIF1AX, EIF4A2, EP300, ERBB3, ERBB4, ERCC5, ESR1, ETV1, ETV6, FANCA, FANCB, FANCC, FANCD2, FANCL, FBXW7, FGFR3, FGFR4, FLT3, FLT4, HGF, HIST1H1C, HIST1H1E, HRAS, INHBA, INPP4B, INSR, IRF4, IRS1, KDR, KMT2A, KMT2B, KMT2C, KMT2D, LATS1, MAP3K1, MAX, MED12, MGA, MLH1, MRE11A, MSH2, MSH3, MSH6, MST1, MST1R, MUTYH, NBN, NOTCH1, NOTCH2, NOTCH3, NOTCH4, NTRK1, NTRK3, MTRKR3, PAK7, PBRM1, PDCD1, PDGFRA, PDGFRB, PIK3C2G, PIK3C3, PIK3CG, PMS1, PMS2, PPM1D, PTCH1, PTPRD, PTPRO, PTPRS, PTPRT, RAD50, RARA, RASA1, RECQL, REL, RET, RPS6KB2, SETD2, SF3B1, SMARCA4, SMARCB1, SMARCD1, SMO, SOCS1, TBX3, TGFBR1, TGFBR2, U2AF1, VHL, XPO1, and ZFHX3, increases the survival score by 1(x) for each gene alteration,
wherein amplification of PDL1 or PDL2 increases the survival score by 1(x) for each gene alteration,
wherein the presence of an amino acid substitution in a gene selected from the group consisting of AKT1, AMER1, APC, ASXL1, AXIN1, AXIN2, BAP1, B2M, CD273, CD274, CTNNB1, CUL3, DNMT1, DNMT3A, DNMT3B, ERRFI2, HLA-A, IL7R, JAK1, JAK2, KEAP1, KRAS, MYC, MYCL, MYCN, NF1, NFE2L2, NFKBIA, PIK3R3, RNF43, SH2D1A, SMAD4, STK11, TCF7L2, TET2, TNFRSF14, and WT1 decreases the survival score by 1(x) for each gene alteration,
wherein amplification of RICTOR decreases the survival score by 1(x) for each gene alteration, and
wherein deletion, loss, frameshift, or truncation of SH2D1A, increases the survival score by 2(x) for each gene alteration;
wherein deletion, loss, frameshift, or truncation of NOTCH, PTEN, or RBM10 decreases the survival score by 1(x) for each gene alteration;
(c) obtaining a survival score of at least 2(x); and
(d) treating the subject with an immunotherapy.

2. The method of claim 1, wherein the immunotherapy comprises a checkpoint inhibitor.

3. The method of claim 2, wherein the checkpoint inhibitor comprises an anti-PD-1 antibody, anti-PD-L1 antibody, anti-CTLA-4 antibody, or a combination thereof.

4. The method of claim 1, wherein the solid tumor is found in a subject with bladder cancer, breast cancer, colorectal cancer, esophagogastric cancer, glioma, head and neck cancer, melanoma, non-small cell lung cancer, renal cell carcinoma, or a combination thereof.

5. The method of claim 1, wherein the at least 70 genes sequenced in step (a) comprise at least AMER1, APC, ATR, ATRX, AXIN1, AXIN2, B2M, BAP1, BLM, BMPR1A, BRD4, CARM1, CD274, CDK8, CDK12, CHEK1, CHEK2, CTNNB1, DNMT3B, DOT1L, DROSHA, EGFR, EIF1AX, EIF4A2, EP300, ESR1, ETV1, ETV6, FBXW7, FGFR3, HIST1H1C, HLA-A, HRAS, INPP4B, IRS1, JAK1, KMT2D, KRAS, MLH1, MUTYH, MYC, MYCN, NFE2L2, NOTCH1, NTRK1, PDCD1, PIK3R3, PMS1, PMS2, POLD1, PTEN, RARA, RASA1, RBM10, RECQL, REL, SF3B1, SH2D1A, SMAD4, SMARCA4, SMARCB1, SOCS1, TBX3, TGFBR1, TGFBR2, TRAF7, WT1, and XPO1.

6. The method of claim 1, wherein the at least 70 genes sequenced in step (a) comprise at least 70 of the genes selected from the group consisting of AKT1, AKT2, AMER1, APC, ARID1A, ARID2, ATM, ATR, ATRX, AURKA, AXIN1, AXIN2, B2M, BABAM1, BAP1, BCL2, BLM, BMPR1A, BRCA2, BRD4, CARM1, CD274, CDK8, CDK12, CHEK1, CHEK2, CTNNB1, CUL3, DDR2, DNMT3A, DNMT3B, DOT1L, DROSHA, EGFR, EIF1AX, EIF4A2, EP300, ERBB3, ERBB4, ESR1, ETV1, ETV6, FANCA, FANCC, FBXW7, FGFR3, HIST1H1C, HLA-A, HRAS, INPP4B, INSR, IRF4, IRS1, JAK1, KMT2D, KRAS, MAP3K1, MLH1, MSH3, MSH6, MUTYH, MYC, MYCN, NF1, NFE2L2, NOTCH1, NOTCH4, NTRK1, PBRM1, PDCD1, PDGFRA, PIK3R3, PMS1, PMS2, POLD1, POLE, PTEN, PTPRS, RARA, RASA1, RBM10, RECQL, REL, SF3B1, SH2D1A, SMAD4, SMARCA4, SMARCB1, SOCS1, TBX3, TGFBR1, TGFBR2, TRAF7, U2AF1, WT1, and XPO1.

7. The method of claim 1, wherein step (a) comprises gene sequencing at least 120 of the genes selected from the group consisting of AMER1, APC, ARID1A, ARID1B, ARID2, ASXL1, ATM, ATR, ATRX, AURKA, AXIN1, AXIN2, B2M, BABAM1, BAP1, BCL2, BCL6, BCOR, BLM, BMPR1A, BRCA2, BRD4, BRIP1, CARM1, CD273, CD274, CDK12, CDK6, CDK8, CHEK1, CHEK2, CSF1R, CTNNB1, CUL3, DDR2, DICER, DNMT1, DNMT3A, DNMT3B, DOT1L, DROSHA, EGFR, EIF1AX, EIF4A2, EP300, ERBB3, ERBB4, ERCC5, ERRF12, ESR1, ETV1, ETV6, FANCA, FANCB, FANCC, FANCD2, FANCL, FBXW7, FGFR3, FGFR4, FLT3, FLT4, HGF, HIST1H1C, HIST1H1E, HLA-A, HRAS, IL7R, INHBA, INPP4B, INSR, IRF4, IRS1, JAK1, JAK2, JUB, KDR, KEAP1, KMT2A, KMT2B, KMT2C, KMT2D, KRAS, LATS1, MAP3K1, MAX, MED12, MGA, MLH1, MRE11A, MSH2, MSH3, MSH6, MST1, MST1R, MUTYH, MYC, MYCL, MYCN, NBN, NF1, NFE2L2, NFKBIA, NOTCH, NOTCH1, NOTCH2, NOTCH3, NOTCH4, NTRK1, NTRK3, PAK7, PBRM1, PDCD1, PDGFRA, PDGFRB, PIK3C2G, PIK3C3, PIK3CG, PIK3R3, PMS1, PMS2, POLD1, POLE, PPM1D, PTCH1, PTEN, PTPRD, PTPRO, PTPRS, PTPRT, RAD50, RARA, RASA1, RBM10, RECQL, REL, RET, RICTOR, RNF43, RPS6KB2, SETD2, SF3B1, SH2D1A, SMAD4, SMARCA4, SMARCB1, SMARCD1, SMO, SOCS1, STK11, TBX3, TCF7L2, TET2, TGFBR1, TGFBR2, TNFRSF14, TRAF7, U2AF1, VHL, WT1, XPO1, and ZFHX3.
